(19) United States
(12) Patent Application Publication
(10) Pub. No.: US 2023/0218335 A1
(43) Pub. Date: Jul. 13, 2023

(54) HIGH-VOLTAGE MINIMALLY INVASIVE APPLICATOR DEVICES FOR SUB-MICROSECOND PULSING

(71) Applicant: PULSE BIOSCIENCES, INC., Hayward, CA (US)

(72) Inventors: Cameron D. HINMAN, Thurmond, NC (US); Kevin L. MOSS, Lathrop, CA (US); David J. DANITZ, San Jose, CA (US)

(21) Appl. No.: 18/000,013

(22) PCT Filed: Jun. 1, 2021
(86) PCT No.: PCT/US2021/035146

§ 371 Date: Nov. 28, 2022

Related U.S. Application Data
(60) Provisional application No. 63/033,813, filed on Jun. 2, 2020.

(51) Int. Cl. A61B 18/14; A61B 18/12

(52) U.S. Cl. CPC A61B 18/1442; A61B 18/1492; A61B 18/1206; A61B 18/1482; A61B 2018/145

(57) ABSTRACT

Described herein are elongate applicator tools adapted to be inserted into a body to deliver high voltage, sub-microsecond electrical energy to target tissue. These tools may be configured as laparoscopes, endoscopes, and/or catheters. Also disclosed herein systems including these tools and method of their operation.

### CROSS REFERENCE TO RELATED APPLICATIONS

This patent application claims priority to U.S. Provisional Patent Application No. 63/033,813 titled “HIGH-VOLTAGE MINIMALLY INVASIVE APPLICATOR DEVICES FOR SUB-MICROSECOND PULSING,” filed on Jun. 2, 2020, which is hereby incorporated by reference in its entirety.

### INCORPORATION BY REFERENCE

All publications and patent applications mentioned in this specification are herein incorporated by reference in their entirety to the same extent as if each individual publication or patent application was specifically and individually indicated to be incorporated by reference.

### FIELD

Described herein are medical apparatuses (e.g., devices, systems, etc.) and methods that may be used to perform medical operations to treat patients. Specifically, the apparatuses described herein can include minimally invasive devices, such as laparoscopes, etc. that may apply high voltage, short electrical pulses to treat patients.

### BACKGROUND

Short, high-field strength electric pulses have been described for electromanipulation of biological cells. For example, electric pulses may be used in treatment of human cells and tissue including tumor cells, such as basal cell carcinoma, squamous cell carcinoma, and melanoma. The voltage induced across a cell membrane may depend on the pulse length and pulse amplitude. Pulses longer than about 1 microsecond may charge the outer cell membrane and may lead to permanent opening of pores. Permanent openings may result in instant or near instant cell death. Pulses shorter than about 1 microsecond may affect the cell interior without adversely or permanently affecting the outer cell membrane and result in a delayed cell death with intact cell membranes. Such shorter pulses with a field strength varying in the range, for example, of 10 kV/cm to 100 kV/cm may trigger apoptosis (i.e. programmed cell death) in some or all of the cells exposed to the described field strength and pulse duration. These higher electric field strengths and shorter electric pulses may be useful in manipulating intracellular structures, such as nuclei and mitochondria. For example, such sub-microsecond (e.g., nanosecond) high voltage pulse generators have been proposed for biological and medical applications.

Because of the high therapeutic voltages, as well as the very fast pulse times, applicators for delivery of such nanopulse energy devices must be configured so as to avoid damaging tissues or otherwise harming the patient. The risks of delivering high voltage energy, such risks including electrical shock, arcing, burns, internal-organ damage, and cardiac arrhythmias, are even more acute when the high voltage device is intended to be inserted into the body.

Thus, it would be beneficial to provide devices, such as laparoscopes, that may apply high voltage, sub-microsecond electrical pulses to treat patients while mitigating the above-mentioned risks.

### SUMMARY OF THE DISCLOSURE

Described herein are apparatuses (including instruments and devices, such as elongate applicator tools, e.g., configured as a laparoscopic device, a catheter, etc. or configured to be introduced or used through a lumen of a laparoscopic device, an endoscope or a catheter) and methods for the treatment of a patient that may use them to more effectively apply therapeutic energy, including but not limited to short, high field strength electric pulses, while minimizing or avoiding the risk of arcing or otherwise harming the tissue. These applicators may be used for minimally invasive procedures, and may be particularly well suited, for example, for treatments of various conditions, disorders and diseases, such as, but not limited to cancer (and other types of abnormal tissue growth), and the like. These applications may be also particularly well suited for use with various fully and partially automated systems, such as robotic systems. In particular, the apparatuses described herein may be configured as apparatuses (e.g., laparoscopic apparatuses) that can be used with a variety of different generator systems, as will be described in greater detail herein.

Thus, the apparatuses described herein may be configured for manual or automated (e.g., robotic-assisted) control. In some examples these apparatuses may be integrated into systems that are configured to be mounted onto or coupled to a movable (e.g., robotic) arm of a robotic system, such as robotic medical treatment system or robotic surgical system. For convenience of description the present disclosure may refer to these as robotic surgical systems, however, it should be understood that such robotic surgical systems are intended to cover any robotic medical treatment system (including for cosmetic applications) and may include robotic systems having guidance. In some examples instruments can be guided and controlled by the robotic surgical system during a surgical procedure. For example, the devices described herein may be used through one or more operating channels of a robotic system.

The apparatuses described herein may include elongate applicator tools that may be manipulated proximally (automatically or manually) to articulate the distal end region (also referred to as tip), including adjusting the angle of the distal end region, the spacing of two or more electrodes (e.g., two sets of electrodes) at the tip, and/or the extension/retraction of the electrodes at the tip. In some examples an elongate applicator tool as described herein includes a proximal handle portion, an elongate body and a distal end region including two or more electrodes. The proximal handle may include one or more controls for manipulating the distal end region of the applicator tool, including articulating the distal end region to change the angle of the distal tip region relative to the elongate body, the rotational position of the distal end region relative to the elongate body, etc. One or more controls (including automatic controls) may also adjust the distance between pairs of electrodes (e.g., cathode and anode) or pairs of sets of electrodes (cathodes and anodes). One or more controls may also or additionally include the extension/retraction of the electrodes (e.g., extending them out of a protected, e.g., insulated, housing on the distal end region).

The elongate applicators may be referred to as elongate applicator tools. The elongate body portion may be rigid, bendable or flexible. In some examples the elongate body portion may be a catheter or catheter body.

According to one aspect, apparatuses described herein comprise medical devices and instruments for use in minimally invasive procedures that are introduced through small incisions and may be equipped with additional tools, such as obturators, cameras, forceps, graspers, etc. In some examples, the apparatuses described herein may be used through a working channel of an endoscope. In some examples, these apparatuses may be configured as a catheter or including an elongate catheter body. In any of the apparatuses or systems described herein the elongate applicators may be configured as a laparoscope (and may be referred to herein as a laparoscope, a laparoscope apparatus, or a laparoscopic instrument). As used herein a laparoscope may, but does not necessarily have to, include one or more visualization components (e.g., a fiber optic, camera, lenses, filters, etc.). Thus, any of the apparatuses described herein may be configured as a scope. An elongate applicator as described herein, including (but not limited to) those configured as a laparoscope, may include a tip having a plurality of electrodes that may be retractable and/or may include a retractable/removable insulating region that may protect and insulate one or more treatment electrodes (e.g., plate or surface electrodes, needle electrodes, knife electrodes, etc.) through which high voltage rapidly pulsed energy may be delivered into the tissue. These apparatuses may be configured safely and reliably to deliver microsecond, nanosecond, picosecond, etc. pulses, and may include an electric field with a pulse width of between 0.1 nanoseconds (ns) and less than 1000 nanoseconds, or shorter, such as 1 picosecond, which may be referred to as sub-microsecond pulsed electric field. This pulsed energy may have high peak voltages, such as 1 to 5 kilovolts per centimeter (kV/cm), 10 kV/cm, 20 kV/cm, 100 kV/cm or higher. Treatment of biological cells may use a multitude of periodic pulses at a frequency ranging from 0.1 per second (Hz) to 10,000 Hz, and may trigger apoptosis, for example, in the diseased tissue or abnormal growth, such as cancerous, precancerous or benign tumors. Selective treatment of such tumors with high voltage, sub-microsecond pulsed energy can induce apoptosis within the tumor cells without substantially affecting normal cells in the surrounding tissue due to its non-thermal nature. A subject may be a patient (human or non-human, including animals). A user may operate the apparatuses described herein on a subject. The user may be a physician (doctor, surgeon, etc.), medical technician, nurse, or other care provider.

Thus, the application of high voltage, fast (e.g., microsecond or sub-microsecond) electrical pulses may include applying a train of electrical pulses having a pulse width, for example, of between 0.1 nanoseconds (ns) and 1000 nanoseconds. Applying high voltage, fast electrical pulses may include applying a train of sub-microsecond electrical pulses having peak voltages of between, for example, 1 kilovolts per centimeter (kV/cm) and 500 kV/cm. Applying high voltage, fast electrical pulses may include applying a train of sub-microsecond electrical pulses at a frequency, for example, of between 0.1 per second (Hz) to 10,000 Hz.

For example, described herein are apparatuses for treating tissue. Any appropriate tissue may be treated, including tissue of one or more organs (e.g., pharynx, esophagus, stomach, small intestine, large intestine, liver, gallbladder, mesentery, pancreas, larynx trachea, bronchia, lungs, diaphragm, kidney, bladder, urethra, ovaries, fallopian tubes, uterus, vagina, testes, epididymis, vas deferens, prostate, bulbourethral glands, pituitary gland, pineal gland, thyroid gland, adrenal glands, heart, arteries, veins, lymph nodes, lymphatic vessel, spleen, thymus, skin, etc.) In some examples the apparatuses and methods described herein may be used to treat one or more of these tissues, as part of a minimally invasive therapy. In some examples the therapy may be for treatment of cancer. In some examples the methods and apparatuses described herein may be used to treat a tumor or tumors, including cancerous, pre-cancerous, benign or non-malignant tumors, lesions or growths.

Any of these apparatuses may be used with a pulse generator. For example, described herein are systems for treating tissue that may include: an elongate applicator tool as described herein (e.g., an elongate body having a distal end region that articulates, from which one or more electrodes are configured to extend), a connector, e.g., a high voltage connector adapted to couple the elongate applicator tool to a pulse generator; and a pulse generator configured to generate a plurality of electrical pulses having amplitude of at least 0.1 kV and a duration of less than 1000 nanoseconds, the pulse generator comprising a port configured to connect to the high voltage connector.

Also described herein are methods of using any of the apparatuses (and systems that include such apparatuses) to treat tissue. Generally the elongate applicator tools described herein may be configured to treat tissue within a body by delivering, through the one or more extendable electrodes, one or a train of high voltage, fast (e.g., sub-microsecond, nanosecond, picosecond) pulses. For example, described herein are methods of treating tissue, the method comprising: inserting a distal end of an elongate applicator tool into a body, wherein the elongate applicator tool comprises at least two electrodes at a distal end region; adjusting (from a proximal control) at least a portion of the distal end region of the elongate applicator tool to adjust an angle and/or separation of at least two electrodes relative to a target tissue, and applying a plurality of electrical pulses having an amplitude of greater than 0.1 kV and a duration of less than 1000 nanoseconds from the at least two electrodes; and delivering the applied plurality of electrical pulses to the target tissue from the at least two electrodes.

For example, described herein are systems for delivering a sub-microsecond pulsed electric field, the system comprising: an elongate applicator tool comprising: an elongate shaft; a distal end region extending from a distal end of the elongate shaft, wherein the distal end region is configured to be controllably articulated relative to the elongate shaft; a proximal handle including a control for selecting articulation of the distal end region; a first electrode and a second electrode on the distal end region; a high voltage connector; and a pulse generator configured to generate a plurality of electrical pulses having amplitude of at least 0.1 kV and a duration of less than 1000 nanoseconds, the pulse generator comprising a port configured to connect to the high voltage connector.

Any of these systems may include a control on the proximal handle to adjust a spacing between the first electrode and the second electrode. The control may be configured to adjust the spacing between the first electrode and the second electrode from between about 0.1 mm (e.g., about 0.2 mm, about 0.3 mm, about 0.4 mm, about 0.5 mm, etc.) to about 1 mm or more (e.g., about 2 mm, about 3 mm, about 4 mm, about 5 mm, about 6 mm, about 7 mm, about 8 mm, about 9 mm, about 10 mm, about 15 mm, about 20 mm, about 25 mm, about 30 mm, etc. or more). In general, a control may be any appropriate input, including a button, dial, slider, knob, trigger, etc. The control may be mechanical or non-mechanical (e.g., electric, magnetic, etc.), including control circuitry, etc. The control may be manual, automatic or semi-automatic. For example, the apparatus may be configured to automatically adjust (via control circuitry) the spacing between the first and second electrodes based on one or more determined parameters. The control may be configured to adjust the spacing to one or more preset distances (e.g., between about 0.1 mm and about 30 mm).

The first and second electrodes may extend distally from the distal end region. Any of these systems may include a control on the proximal handle to extend and retract the first and second electrodes from a housing.

As mentioned above, any of the elongate applicator tools described herein may be configured as a laparoscope. Thus, any of these elongate applicator tools may include an imaging element (e.g., fiber optic, camera, etc.) on the distal end region providing a visual output. In some examples the apparatus may include a light source. In some examples the apparatus may include a connector for connecting to one or more image processors.

The distal end region may include a first jaw and a second jaw configured to open and close and a control on the proximal handle configured to control opening and closing of the first jaw and the second jaw. The first electrode may extend distally from the first jaw and the second electrode extends distally from the second jaw. The first jaw and the second jaw may be configured to open in parallel. In some examples a jaw spacing control on the proximal handle may be configured to set a jaw spacing between the first jaw and the second jaw in a closed configuration.

In general, any of the methods and apparatuses described herein may be configured to determine (e.g., detect, sense, measure, etc.) the spacing or distance between the electrodes (e.g., the first and second electrodes, or a first set of electrodes and a second set of electrodes), where the spacing between the electrodes may be adjusted, such as (but not limited to) by adjusting the distance between jaws or arms on which the electrodes are coupled. The distance or separation between the electrodes may be detected by any appropriate component, such as a sensor, a mechanical detector (e.g., measuring angle, separation distance, etc.) including measuring by pre-calibration (such as correlating a known position of a control controlling the jaw position corresponds to a known distance between the electrodes, etc.), an optical detector (e.g., an IR detector, photodetector, etc.), magnetic sensor (e.g., a hall effect sensor), etc. For example, any of these apparatuses may include a sensor configured to detect a jaw spacing distance (and/or electrode spacing distance) such as between the first jaw and the second jaw. Examples of appropriate sensors or detectors may include: a rotary potentiometer, a linear variable differential transformer (LVDT), a proximity sensors (e.g., inductive, capacitive, ultrasonic, infrared, etc.), a linear optical encoder, and/or a rotary optical encoder. Alternatively or additionally, any of these methods may include setting, either manually or automatically, the spacing between the electrodes, by controlling the spacing of the jaws on which the electrodes are positioned. The control may be calibrated, so that a predetermined actuation of the control may adjust the spacing between the electrodes (or sets of electrodes) by a known distance per unit of actuation.

In general, these apparatuses may be configured for use with a robotic arm or robotic control (e.g., providing automatic or automated robotic control of operation of the apparatus). For example, the proximal handle may be configured to be coupled to a robotic arm for computer controlled activation and/or positioning of the first and second electrodes.

Also described herein are apparatuses for delivering a high voltage, sub-microsecond pulsed electric field, the apparatus comprising: an elongate shaft; a distal end region comprising a first jaw and a second jaw, the distal end region extending from a distal end of the elongate shaft; a first electrode on the first jaw; a second electrode on the second jaw; a proximal handle including a control for opening and closing the first jaw and the second jaw; and a sensor configured to output an electrode spacing distance between the first electrode and the second electrode. The distal end region may be configured to be controllably bent to a bend angle relative to the elongate shaft and wherein the proximal handle comprises a control for selecting the bend angle.

The first electrode and the second electrode may be needle electrodes, plate electrodes, surface electrodes, etc. The first electrode may extend distally from the first jaw and the second electrode may extend distally from the second jaw.

In general, any of these apparatuses may be used in conjunction with or be a part of the system that includes a pulse generator configured to generate a plurality of electrical pulses having amplitude of at least 0.1 kV and a duration of less than 1000 nanoseconds, the pulse generator may be configured to connect to the apparatus with a high voltage connector. The pulse generator may comprise a controller configured to receive a spacing distance between the first electrode and the second electrode from the sensor, and to adjust an applied pulsed electric field based on an electrode spacing distance between the first electrode and the second electrode.

Any of these apparatuses may include a suction inlet adjacent to the first and second electrodes.

As mentioned, any of these apparatuses may be configured so that the proximal handle is adapted to be coupled to a robotic arm, for example, for computer controlled activation of the first and second electrodes.

Also described herein are apparatuses for delivering a high voltage, sub-microsecond pulsed electric field, the apparatus comprising: an elongate shaft; a distal end region extending from a distal end of the elongate shaft and configured to be controllably bent to a bend angle relative to the elongate shaft; a first electrode on the distal end region; a second electrode on the distal end region; a proximal handle including a spacing control for adjusting the spacing between the first electrode and the second electrode; and an articulation control on the proximal handle configured to adjust the bend angle of the distal end region. Any of these apparatuses may include a sensor configured to output an electrode spacing distance between the first electrode and the second electrode. The spacing control may be configured to adjust the spacing between the first electrode and the second electrode from between about 0.5 mm to 1 mm or more. The first and second electrodes may extend distally from the distal end region. As mentioned, the elongate applicator may be configured as a laparoscope, endoscope or a catheter.

Any of these apparatuses may include a control on the proximal handle to extend and retract the first and second electrodes from a housing of the distal end region.

The distal end region may comprise a first jaw and a second jaw configured to open and close to adjust the spacing between the first electrode and the second electrode. For example, the first electrode may extend distally from the first jaw and the second electrode extends distally from the second jaw. The first jaw and the second jaw may be configured to open in parallel.

Any of these apparatuses may include a jaw spacing control on the proximal handle configured to set a jaw spacing between the first jaw and the second jaw in a closed configuration.

Also described herein are systems for delivering a high voltage, sub-microsecond pulsed electric field, the system comprising: elongate applicator tool comprising: an elongate shaft; a distal end region comprising a pair of jaws, the distal end region extending from a distal end of the elongate shaft, wherein the distal end region is configured to be controllably bent to a bend angle relative to the elongate shaft; a proximal handle including a control for selecting the bend angle of the distal end region and a control for opening and closing the pair of jaws; a first electrode on a first jaw of the pair of jaws; a second electrode on a second jaw of the pair of jaws; and a sensor configured to output an electrode spacing distance between the first electrode and the second electrode; and a pulse generator configured to generate a plurality of electrical pulses having amplitude of at least 0.1 kV and a duration of less than 1000 nanoseconds, the pulse generator comprising a port configured to connect to the elongate applicator tool, wherein the pulse generator is configured to receive the electrode spacing distance between the first electrode and the second electrode from the sensor, and to adjust an applied pulsed electric field based on the electrode spacing distance between the first electrode and the second electrode.

Also described herein are methods of using any of these apparatuses and systems. For example, a method may include: contacting a distal end of an elongate applicator tool against a target tissue, wherein the elongate applicator tool comprises a first electrode and a second electrode at a distal end region; detecting, on the elongate applicator tool, a separation distance between the first and second electrode; determining, based at least in part on the separation distance between the first and second electrode, an energy to be applied to the target tissue in a plurality of electrical pulses having an amplitude of greater than 0.1 kV and a duration of less than 1000 nanoseconds, to treat the tissue; and delivering the energy to the target tissue.

The method may include transmitting the separation distance to a pulse generator. Any of these methods may include connecting the elongate applicator tool to a pulse generator using a high voltage connector.

In some examples the methods may include inserting the elongate applicator tool into the body. For example, the method may include contacting the distal end of the elongate applicator tool against the target tissue comprises placing the target tissue between a first jaw and a second jaw of the elongate applicator tool, wherein the first electrode is on the first jaw and the second electrode is on the second jaw. In some examples, the method includes compressing target tissue between the first jaw and the second jaw.

Any of these methods may include adjusting or allowing an adjustment of a control on the elongate applicator tool to set a closing distance between the first jaw and the second jaw. Adjusting the separation distance between the first electrode and the second electrode may be done before detecting the separation distance between the first and second electrode or after. Detecting may comprise optically detecting the separation distance between the first and second electrode from an optical sensor on the elongate applicator tool. In some examples detecting comprises electrically detecting the separation distance between the first and second electrode.

Delivering the energy to the target tissue may comprise applying suction to seal the first and second electrodes to the target tissue while delivering the energy.

In some implementations, a method may include: inserting a elongate applicator tool into a body; contacting a distal end of the elongate applicator tool against a target tissue by placing the target tissue between a first jaw and a second jaw of the elongate applicator tool, wherein the elongate applicator tool comprises a first electrode on the first jaw and a second electrode on the second jaw; detecting, on the elongate applicator tool, a separation distance between the first and second electrode; determining, based at least in part on the separation distance between the first and second electrode, an energy to be applied to the target tissue in a plurality of electrical pulses having an amplitude of greater than 0.1 kV and a duration of less than 1000 nanoseconds, to treat the target tissue; and delivering the energy to the target tissue.

Also described herein are methods of operating of the tool or a system, as well as methods that may be performed without contacting or manipulating any tissue. Some of the methods may be used for, for example, for calibrating the device, or for repairing the device, or for adjusting configuration or positioning of the device. For example, describe herein are methods comprising: adjusting a first proximal control of an elongate applicator tool to change a separation distance between a first electrode and a second electrode, wherein the first electrode and the second electrode extend from a distal end region of the elongate applicator tool; adjusting a second proximal control to articulate the distal end region relative to a shaft of the elongate applicator tool; receiving, in a controller, a separation distance between the first electrode and the second electrode; and determining based at least in part on the separation distance between the first and second electrode, an energy to be delivered between the first and second electrodes. Determining may comprise determining the energy to be delivered between the first and second electrodes in a plurality of electrical pulses having an amplitude of greater than 0.1 kV and a duration of less than 1000 nanoseconds.

The method may include transmitting the separation distance to a pulse generator, wherein the controller comprises part of the pulse generator. The method may also include connecting the elongate applicator tool to a pulse generator using a high voltage connector. Adjusting the spacing may comprise adjusting a distance between a first jaw and a second jaw of the elongate applicator tool, wherein the first electrode is on the first jaw and the second electrode is on the second jaw. Any of these methods may include adjusting a separation control on the elongate applicator tool to set a minimum closing distance between the first jaw and the second jaw. The method may also include actuating a third proximal control to extend the first electrode and the second electrode from a housing on the distal end region.

In some examples, the method includes detecting the separation distance between the first electrode and the second electrode. For example, the method may include optically detecting the separation distance between the first and second electrode using an optical sensor on the elongate applicator tool. In some examples the method may include electrically detecting the separation distance between the first and second electrode.

The methods and apparatuses described herein may be related to, and may be used with, any of the methods and apparatuses described in U.S. Pat. No. 10,543,357 for “HIGH VOLTAGE CONNECTORS AND ELECTRODES FOR PULSE GENERATORS”, and to U.S. application Ser. No. 15/920,389, titled “TREATMENT INSTRUMENT AND HIGH-VOLTAGE CONNECTORS FOR ROBOTIC SURGICAL SYSTEM,” filed on Mar. 13, 2018, which are each hereby incorporated by reference in their entirety for all purposes.

### BRIEF DESCRIPTION OF THE DRAWINGS

The novel features of the present disclosure are set forth with particularity in the claims that follow. A better understanding of the features and advantages of the present disclosure will be obtained by reference to the following detailed description that sets forth illustrative embodiments, in which the principles of the disclosure are utilized, and the accompanying drawings of which:

FIG. 1 illustrates one example of a system, including an elongate applicator tool of the present disclosure and a pulse generator, for delivery of high voltage, fast pulsed electrical energy.

FIG. 2 illustrates an example of pulse profiles for voltage and current that may be applied by the apparatuses described herein.

FIGS. 3A and 3B schematically illustrate examples of side-facing, penetrative electrodes arranged to deliver high voltage (e.g., 15 kV), sub-microsecond pulses of electrical energy into tissue from the distal end of an elongate applicator tool.

FIG. 3C illustrates one example of voltage versus time plot for a system delivering sub-microsecond pulses of electrical energy.

FIGS. 3D and 3E schematically illustrate examples of side-facing, non-penetrative electrodes arranged to deliver high voltage (e.g., 15 kV), sub-microsecond pulses of electrical energy into tissue from the distal end of an elongate applicator tool.

FIGS. 4A-4B illustrate a distal end region of one example of a high voltage elongate applicator tool as described herein. FIG. 4A shows the elongate applicator tool with the electrodes retracted; FIG. 4B shows the elongate applicator tool with the electrodes extended.

FIGS. 4C-4D illustrate a schematic cut-away distal end region of the elongate applicator tool of FIGS. 4A-4B. FIG. 4C shows electrodes retracted and FIG. 4D shows electrodes extended.

FIGS. 5A-5B show another example of a distal end region of an elongate applicator tool having distal jaws that open and close with electrodes on the distal jaws configured to apply high voltage, sub-microsecond pulses between the jaws.

FIGS. 5C-5D shows alternative views of the elongate applicator tool of FIGS. 5A-5B.

FIG. 6 is a cut-away view illustrating one example of a proximal handle of an elongate applicator tool, including multiple control elements for manipulating the distal end region of a high voltage elongate applicator tool.

FIGS. 7A-7C are cut-away views illustrating various positions in operation of a proximal handle of one example of a high voltage elongate applicator tool such as the one shown in FIG. 6 to change the relative angle (articulation) of the distal end region (e.g., the two or more electrodes) of the elongate applicator tool.

FIGS. 8A-8B are cut-away views illustrating another example of operation of a proximal handle of a high voltage elongate applicator tool to open and close the jaws of the elongate applicator tool.

FIGS. 9A-9D are various cut-away views illustrating an example of a jaw closure limiter, regulating (e.g., limiting) how close (in separation distance) the distal jaws may be moved during operation of a high voltage elongate applicator tool.

FIGS. 10A-10D are close-up exterior views illustrating operation of a distal end region of another example of a high voltage elongate applicator tool.

FIGS. 11A-11C are schematic representations showing one example of part of a mechanism for operation of jaws of a high voltage elongate applicator tool that may be actuated by control cables from an elongate applicator tool proximal control. FIG. 11A shows a side view with the jaws closed. FIG. 11B shows a top cross-section view and FIG. 11C shows side view with the jaws open. Two jaw elements each having a pivot pin/hole and cam pin receiving slot as used in the jaw mechanism are shown in this example.

FIGS. 12A-12D illustrate an example of one example of the operative elements of a handle of an instrument that may assist in controlling articulation of the distal end region of the instrument. FIG. 12A is a perspective exploded schematic diagram showing the operative elements of the handle assembly and connection paths for the pieces. FIGS. 12B-12D illustrate the articulation of a distal end region of the device by moving the proximal handle portion. In FIG. 12B the handle portion is bent down relative to the elongate shaft of the device, causing the distal end indicator to bend up relative to the shaft. In FIG. 12C the handle portion is in line with the shaft, and the distal end indicator is also in line with the shaft. In FIG. 12D the handle portion is bent up relative to the elongate shaft of the device, causing the distal end indicator to bend down relative to the shaft.

FIG. 13A is perspective schematic diagram showing an example of an articulated distal end region of the elongate applicator tool in-line with a proximal handle illustrating an example of how the controls in the handle may cause extension of the electrodes from the distal end of the elongate applicator tool.

FIGS. 13B and 13C are back side view perspective diagram schematically illustrating the connection between the operative elements of the handle and the distal end region or tip of the elongate applicator tool. FIG. 13B shows electrodes of the tip fully extended and FIG. 13C shows electrodes of the tip retracted.

FIGS. 14A-14G represent another example of the operative elements of the handle of the instrument that assist in controlling articulation of the distal end region or the tip of the instrument. FIG. 14A is an example of a perspective exploded diagram showing operative elements of the assembly of the handle. FIGS. 14B-14D show examples of the up, down and generally aligned positions of the operative elements of the control mechanisms in the handle for rotating and opening/closing actions of the jaws at the distal end region of the elongate applicator tool. FIGS. 14E-14G show examples of the operative elements of an apparatus configured as shown in FIG. 14A in which the distal end region of the apparatus may be selectively controlled to rotate a first portion relative to a second portion by a proximal control.

FIG. 15 is a perspective cut-away view illustrating one example of a proximal handle of an elongate applicator tool such as the one shown in FIGS. 12A-12D.

FIGS. 16A-16D is a schematic representation showing the operation of another example of a portion of a distal end region of a high voltage, sub-microsecond elongate applicator tool including a tissue-grasping set of jaws and set of electrodes.

FIGS. 17A-17C illustrate yet another example of a distal end region of a high voltage elongate applicator tool including discrete dimensional step adjustment ratcheting engagement of a pair of plate electrodes on the clamping jaws.

FIGS. 18A-18B show another example of a distal end region of a high voltage elongate applicator tool including a pair of plate electrodes on the clamping jaws.

FIGS. 19A-19B show another example of a distal end region of a high voltage elongate applicator tool including continuous non-stepped dimensional adjustable ratcheting engagement of pairs of electrodes on the clamping jaws.

FIG. 20A illustrates one example of a high voltage elongate applicator tool configured to apply suction to help maintain contact with a target tissue as energy is applied.

FIGS. 20B-20C illustrate enlarged views of the distal, articulating end region of the elongate applicator tool of FIG. 20A.

FIGS. 21A-21C illustrate operation of another example of a high voltage elongate applicator tool including vacuum assist similar to that shown in FIG. 20A. FIG. 21A shows the device with the distal end region articulated in a first direction, FIG. 21B shows the distal end region in a linear configuration, while FIG. 21C shows the device with the distal end region articulated in a second direction, opposite from the first direction.

FIG. 22A illustrates one example of an articulating distal end region of a high voltage elongate applicator tool similar to that shown in FIG. 21A-21C.

FIGS. 22B-22C illustrate the construction of an articulating distal end region shown in FIG. 22A.

FIG. 23 schematically illustrates an example of a method that can be used with various examples of the applicator tools of the present disclosure, such as a method of treating a vocal cord lesion (e.g. polyp) as described herein.

FIG. 24 schematically illustrates an example of a method of treating endometriosis as described herein.

FIG. 25 is an example of a distal end region of an elongate applicator apparatus having distal jaws that open and close with side-facing electrodes on the distal jaws that are configured to apply high voltage, sub-microsecond pulses.

FIG. 26 is another example of a distal end region of an elongate applicator apparatus having distal jaws that may open and close to adjust the spacing between the side-facing electrodes.

### DETAILED DESCRIPTION

Described herein are elongate applicator tools adapted to be inserted into a body to deliver high voltage (e.g., microsecond, nanosecond, picosecond, etc.) electrical energy to target tissue without damaging surrounding (non-target) tissue. These elongate applicator tools may be also referred to herein as elongate applicator apparatus or instrument.

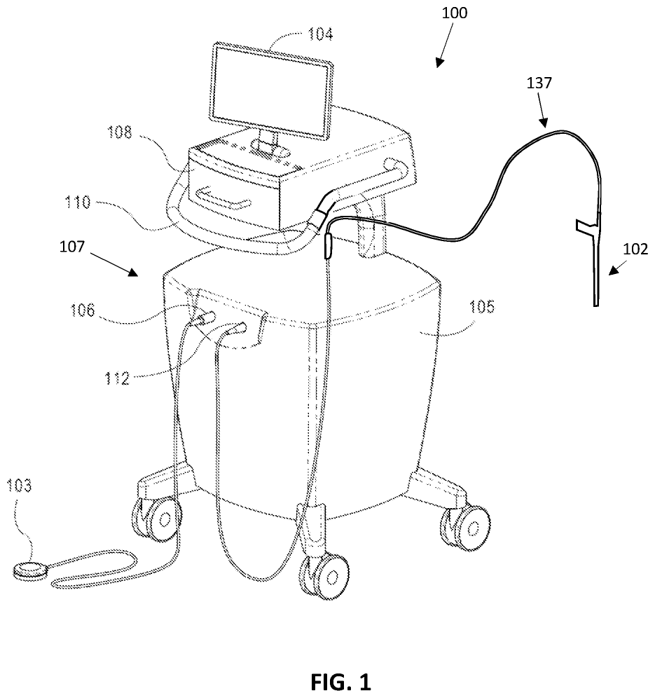

FIG. 1 illustrates one example of a system 100 (also referred to herein as a high voltage system or a sub-microsecond generation system) for delivering high voltage, fast pulses of electrical energy that may include a elongate applicator tool 102, a pulse generator 107, footswitch 103, and user interface 104. The system 100 may provide the high voltage electrical energy pules to treat tissues, including tissues of one or more organs (e.g., pharynx, esophagus, stomach, small intestine, large intestine, liver, gallbladder, mesentery, pancreas, larynx trachea, bronchia, lungs, diaphragm, kidney, bladder, urethra, ovaries, fallopian tubes, uterus, vagina, testes, epididymis, vas deferens, prostate, bulbourethral glands, pituitary gland, pineal gland, thyroid gland, adrenal glands, heart, arteries, veins, lymph nodes, lymphatic vessel, spleen, thymus, skin, etc.) In some examples the apparatuses and methods described herein may be used to treat one or more of these tissues, as part of a minimally invasive therapy. In some examples the therapy may be for treatment of cancer. In some examples the methods and apparatuses described herein may be used to treat a tumor or tumors, including cancerous, pre-cancerous, benign or non-malignant tumors, lesions or growths. In some other examples, the methods and apparatuses described herein may be used to treat any feasible tissue or cell.

Footswitch 103 is connected to housing 105 (which may enclose the electronic components) through a cable and connector 106. The elongate applicator tool 102 may include electrodes and is connected to housing 105 and the electronic components therein through a cable 137 and high voltage connector 112. The high voltage system 100 may also include a handle 110 and storage drawer 108. The system 100 may also include a holder (e.g., holster, carrier, etc.)(not shown) which may be configured to hold the elongate applicator tool 102.

A human operator may select a number of pulses, amplitude, pulse duration, and frequency information, for example by inputting such parameters into a numeric keypad or a touch screen of interface 104. In some examples, the pulse width can be varied. A microcontroller may send signals to pulse control elements within system 100. In some examples, fiber optic cables are used which allow control signaling while also electrically isolating the contents of the metal cabinet with sub-microsecond pulse generation system 100, e.g., the high voltage circuit, from the outside. In order to further electrically isolate the system, system 100 may be battery powered instead of being powered from a wall outlet.

The elongate applicator tool 102 may be hand-held (e.g., by a user) or it can be affixed to a movable arm of a robotic system, and its operation may be at least partially automated or fully automated, including computer-controlled.

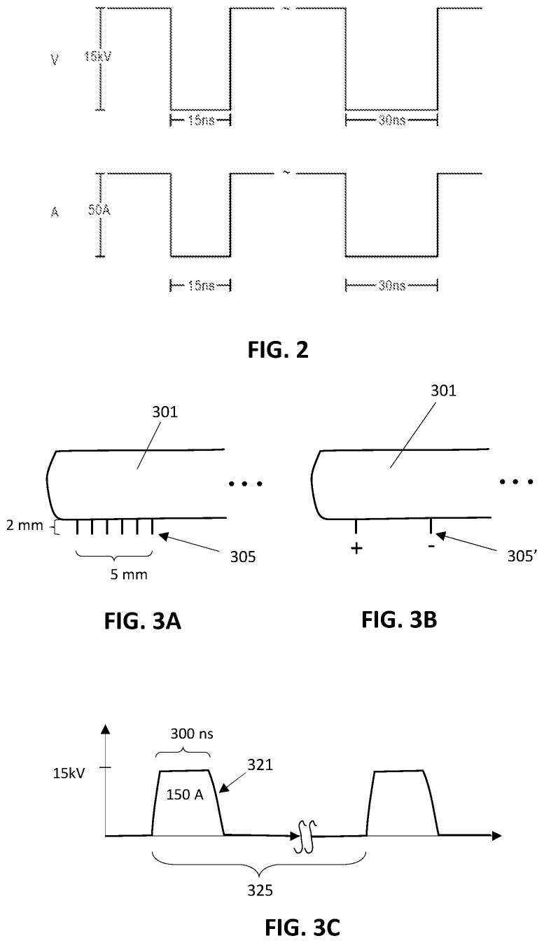

FIG. 2 illustrates a pulse plot output profiles for both voltage and current for high voltage, sub-microsecond pulsing. In the example of FIG. 2, output from the system 100 includes a first and second pulse, with the voltage plot shown in the top portion of the figure and the current plot shown on the bottom portion of the figure. In this example, the first pulse has an amplitude of about 15 kV, a current of about 50 A, and a duration of about 15 ns. The second pulse has an amplitude of about 15 kV, a current of about 50 A, and a duration of about 30 ns. Thus, in some examples, 15 kV may be applied to electrodes connected to the system having 4 mm between the plates so that the target tissue experiences 37.5 kV/cm (e.g., 15 kV/0.4 cm), and current between 12 and 50 A. Given a voltage, current depends heavily on the electrode type and tissue resistance.

While FIG. 2 illustrates a specific example, other pulse profiles may also be generated. For example, in some examples, rise and/or fall times for pulses may be less than 20 ns, about 20 ns, about 25 ns, about 30 ns, about 40 ns, about 50 ns, about 60 ns, about 75 ns, or greater than 75 ns. In some examples, the pulse voltage may be less than 5 kV, about 5 kV, about 10 kV, about 15 kV, about 20 kV, about 25 kV, about 30 kV, or greater than 30 kV. In some examples, the current may be less than 10 A, about 10 A, about 25 A, about 40 A, about 50 A, about 60 A, about 75 A, about 100 A, about 125 A, about 150 A, about 175 A, about 200 A, or more than 200 A. In some examples, the pulse duration may be less than 10 ns, about 10 ns, about 15 ns, about 20 ns, about 25 ns, about 30 ns, about 40 ns, about 50 ns, about 60 ns, about 75 ns, about 100 ns, about 125 ns, about 150 ns, about 175 ns, about 200 ns, about 300 ns, about 400 ns, about 500 ns, about 750 ns, about 1 μs, about 2 μs, about 3 μs, about 4 μs, about 5 μs, or greater than 5 μs.

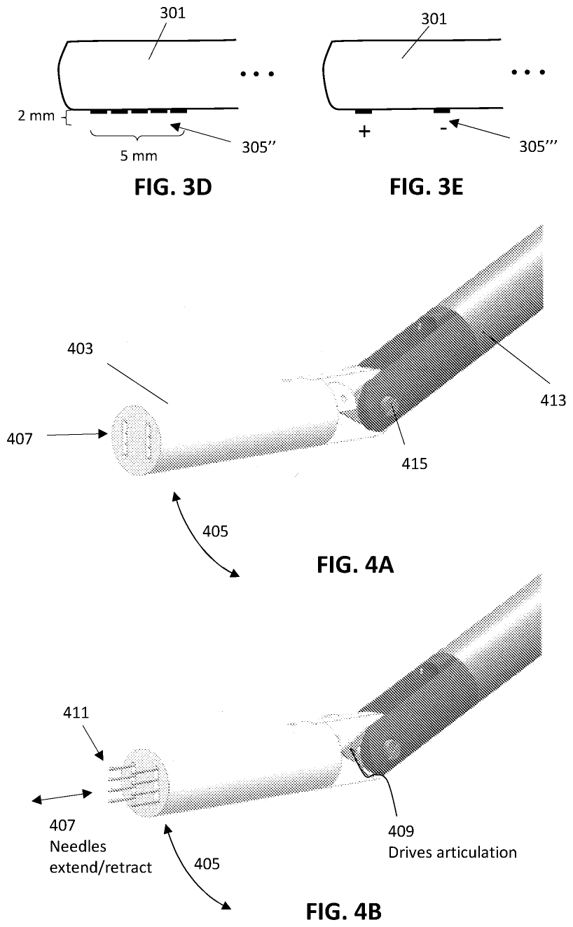

The electrodes described herein may be side-facing electrodes. For example, FIGS. 3A and 3B schematically illustrates examples of side-facing electrodes that may be present at the distal end of a device 301 (e.g., a portion of an elongate applicator tool) shown as perpendicularly extending or extendable needle or knife electrodes that may penetrated into tissue to be treated. In FIG. 3A, the exemplary electrodes 305 extend 2 mm from the apparatus into the tissue to be treated and form a row that is about 5 mm long. More than one row of electrodes (arranged, e.g., along the long axis of the distal end of the elongate applicator tool and/or at an angle, including 90 degrees to the long axis) may be provided. For example, multiple (e.g., two, three, etc.) rows of electrodes, e.g., 0.5 mm long electrodes, 1 mm long electrodes, 1.5 mm long electrodes, 2 mm long electrodes, etc. may be provided. Further, the space between electrodes may be less than or greater than 1 mm (e.g., 0.5 mm, 1 mm, 1.5 mm or greater, 2 mm or greater, 2.5 mm or greater, 3 mm or greater, 3.5 mm or greater, 4 mm or greater, 4.5 mm or greater, 5 mm or greater, etc.). For example, FIG. 3B shows an example of a portion of a device 301 having a pair of extending/extendable electrodes 305′ that are separated by 5 mm or more. In some examples the electrodes may be separated by an insulating barrier (border, ring, etc.) between, around and/or adjacent to the electrodes. As will be described herein, in some examples different electrodes may be positioned on different arms (or “jaws”) of an elongate applicator tool.

In general, electrodes may be penetrating (e.g., needle electrodes, knife electrodes, etc., also referred to herein a penetrative electrodes) or non-penetrative (e.g., plate electrodes, etc., also referred to herein as non-penetrative electrodes). For example, the electrodes shown schematically in FIGS. 3A and 3B as needle electrodes may be non-penetrating electrodes. In FIGS. 3D and 3E the electrodes 305″, 305′″ are non-penetrating (e.g., plate electrodes). As in FIG. 3A, in FIG. 3D, the distal end of the device 301 includes a plurality of electrodes 305″, shown in the example as a non-penetrating surface or plate electrodes that may be used to deliver a pulsed electric field (e.g., microsecond or sub-microsection pulses) to a tissue. For example, one or more side facing non-penetrating electrodes may be disposed on the side of the distal end of the device 301 so that adjacent electrodes are spaced apart. A set or array of electrodes that are spaced apart may be used.

In one example, an apparatus having side facing, non-penetrating electrodes (e.g., plate electrodes) may be used to contact tissue of the female urogenital system (e.g., ovaries, fallopian tubes and the tissue lining the pelvis) to treat endometriosis. For example, an apparatus configured as a paddle or laparoscopic tissue retractor including electrodes as described herein may be used to treat endometriosis. The apparatus may be expanded out and may be pushed against the abnormal endometrial lining to treat endometriosis. When treating endometriosis using the methods and apparatuses described herein, the target tissue to be treated may be referred to as abnormal endometrium. Abnormal endometrium includes tissue similar to the endometrium (that normally lines the inside of the uterus) which grows outside of the uterus, most commonly involving the ovaries, fallopian tubes and the tissue lining the pelvis. Abnormal endometrium may also be referred to as abnormal endometrial lining or endometrial-like tissue.

In some examples, side facing non-penetrating electrodes as described herein may be used to contact and treat different conditions, for example, one or more lesions (e.g., growths, such as nodules, polyps and/or cysts) on a vocal fold (e.g., vocal cord) within the body. For example, an apparatus configured as a paddle or laparoscopic tissue retractor including electrodes as described herein may be expanded out and may be pushed against tissue of the vocal fold to treat a vocal lesion.

Unless the context makes clear otherwise, any of the examples showing penetrative electrodes described herein may be configured to use non-penetrative electrodes, and vice-versa.

FIG. 3C shows another example of a plot of pulse train that may be delivered by a system (e.g., high voltage, fast pulsing electrical generator and elongate applicator tool for delivery thereof). In particular, FIG. 3C shows an example of a voltage vs. time plot for sub-microsecond pulsing showing a 15 kV peak (at 150 amps) for pulses 321 of 300 ns. The pulses may be repeated at a desired repetition rate (interval 325), such as, e.g., between 0.1 Hz and 25 kHz or more. Thus, systems described herein may include, in addition to the instrument (e.g., the elongate applicator tool), a pulse generator such as the one shown schematically in FIG. 1, configured to emit pulses in the sub-microsecond range, similar to the output parameters described above.

In general, the systems of the present disclosure may comprise additional elements, such as power supplies, and/or a high voltage connector for safely connecting the elongate applicator tool device to a high voltage power source. As described above, these systems and devices are configured to apply high voltage, sub-microsecond pulsed electrical energy.

The high voltage pulsing elongate applicator tools may be any appropriate length (e.g., between 6 inches and 200 inches, e.g., between 7 inches and 150 inches long, etc.) and may have any appropriate outer diameter, including, but not limited to between 1 French (Fr), e.g., ⅓ mm and 34 Fr (e.g., 11.333 mm) (between 3 Fr and 30 Fr, between 4 Fr and 15 Fr, 30 Fr or less, 25 Fr or less, 22 Fr or less, 20 Fr or less, 18 Fr or less, 16 Fr or less, 15 Fr or less, 14 Fr or less, 12 Fr or less, 10 Fr or less, 9 Fr or less, 8 Fr or less, etc.).

In general a elongate applicator tool as described herein (e.g., a high voltage, sub-microsecond elongate applicator tool) may include an elongate body with a distal end region including one or more electrodes and/or pairs of electrodes, and a proximal end including a handle and one or more controls (e.g., control elements or mechanisms) for manipulating the distal end region of the elongate applicator tool. As used herein, the terms handle, handle housing, handpiece, and the like may be used to describe a proximal portion of the elongate applicator tool and are not necessarily meant to indicate hand-held device. Any of the handles described herein may be configured to be hand-held and may include a grip region, for example. Alternatively or additionally, the elongate applicator tool may be coupled to and/or controlled by endoscope or a robotic arm or any other feasible device. The distal end region of the high voltage, sub-microsecond elongate applicator tool may be configured to allow manipulation of the portion of the elongate applicator tool including the one or more electrodes. For example, the distal end region or tip that can be articulated or moved (e.g., one or more of: up/down, in/out, rotating clockwise/counterclockwise, etc.). Separately or in conjunction, the one or more electrodes may be configured to move (e.g., to extend and/or retract) from the distal end of the tip. In some examples, the distal end region may be configured to include a pair of jaws for grasping tissue. The one or more electrodes (and/or one or more pairs of electrodes) may be present on the jaws and/or they may be separate from the jaws.

In some examples the high voltage, sub-microsecond elongate applicator tool may include a pair of parallel jaws, or substantially parallel jaws, meaning that the jaws may be parallel through majority of the movement of the jaws, but may be configured to assume a non-parallel orientation when nearly fully opened and/or closed.

Any of the elongate applicator tools described herein may include one or more electrodes of any configuration, including needle electrodes, surface electrodes, plate electrodes, knife electrodes, etc. In some examples these electrodes may be variable depth needle electrodes that can penetrate tissue to a selectable variable depth. The electrodes may be on either a straight non-articulating shaft or in some examples, on an articulating shaft, including on one or more jaws at the distal end of the shaft of the elongate applicator tool. In some examples, the elongate applicator tool includes jaws configured as a grasper to hold the tissue while deploying the needles. The deployable electrodes in some examples may be retractable needle electrodes. For example, in some examples of the elongate applicator tools described herein the needles may extend out the distal end of the tip.

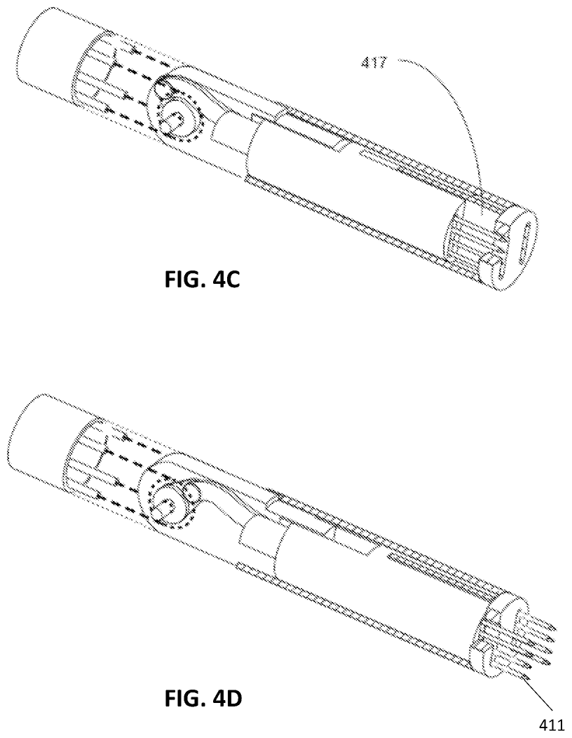

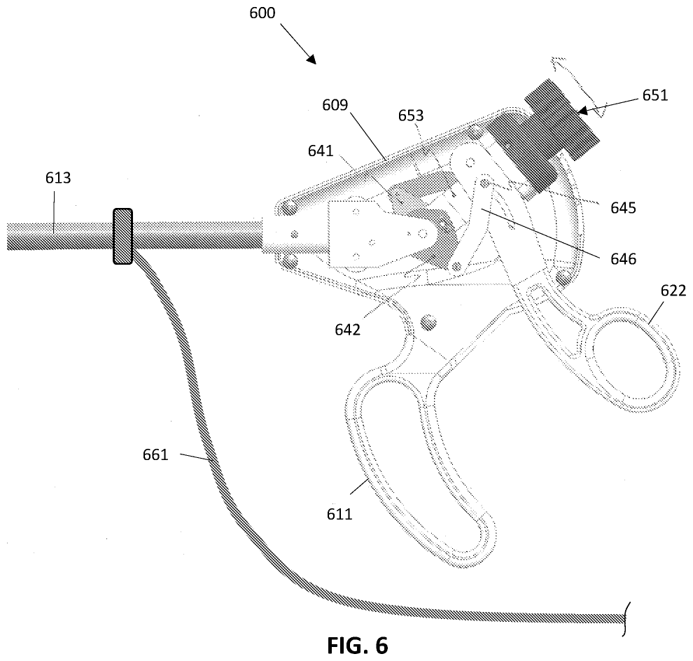

For example, FIGS. 4A-4B illustrate one example of the elongate applicator tool of the present disclosure that includes an articulating distal end region or tip 403 that is hinged (at a hinge point 415) to pivot (e.g., articulate up/down or side-to-side 405) from the distal end of the elongate shaft 413 of the elongate applicator tool. The elongate applicator tool of FIGS. 4A-4D may be configured with a rigid shaft to be used as a laparoscopic instrument. In this embodiment, the elongate applicator tool may be inserted through a canula. In addition, the elongate applicator tool of FIG. 4A-4D may be configured with a flexible shaft to be used as an endoscopic instrument or catheter. In such embodiments, the elongate applicator tool may be inserted through the working channel of an endoscope or catheter sheath. In this example, the tip 403 of the elongate applicator tool can articulate in one degree of freedom and includes a plurality of needle electrodes 411 that can be extended and retracted from the distal end 407. In FIG. 4A the electrodes are retracted; in FIG. 4B the electrodes are extended. The operator may control the articulation of the tip 403 and also the extending/retracting of the electrodes. The proximal end of the elongate applicator tool may include a handle or hand piece (shown, for example, in FIG. 6, below) that may be used to control this motion. Examples of ways in which the elongate applicator tool may be configured to articulate the distal end region as well as extend/retract the needle electrodes is described in greater detail below. For example, in FIGS. 4A-4B the apparatus may include an articulation mechanism 409 (e.g., an articulating puller/pusher, tendon, cable, wire, rod, etc.) that drives articulation of the distal end region. A separate puller/pusher (e.g., an electrode puller/pusher, which may be a tendon, cable, pulley, wire, rod, etc.) may also be used to extend and/or retract the electrodes. In some examples the needle electrodes may be independently extended or retracted relative to the articulation of the distal end region.

FIGS. 4C and 4D show cut-away views of a distal end region of one example of an elongate applicator tool as described herein. In FIG. 4C the electrodes (needle electrodes) are not visible, as they are retracted into the distal end 407 of the articulating tip 403; the electrodes (needle electrodes 411 are shown extended in FIG. 4D. An insulating wall 417 inside the needle holder may be positioned between the needles to prevent arcing or short circuiting should the needles be activated when they are in a less than fully extended position. The extension and retraction of the electrodes may be controlled by a control (electrode extension control), for example, on the proximal end of the elongate applicator tool, including on the handle. In some examples, the extension and retraction of the electrodes may be controlled by a processor, state machine, embedded controller or other similar device.

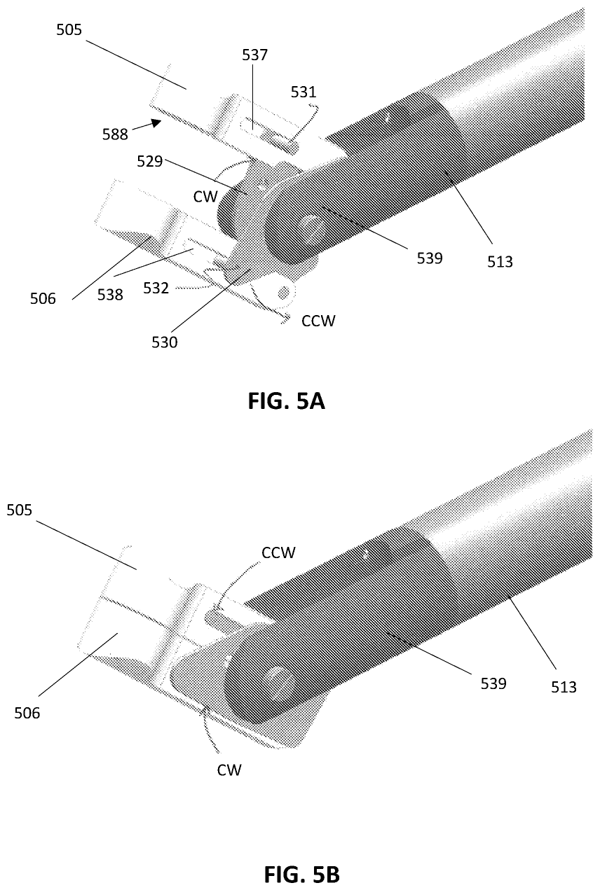

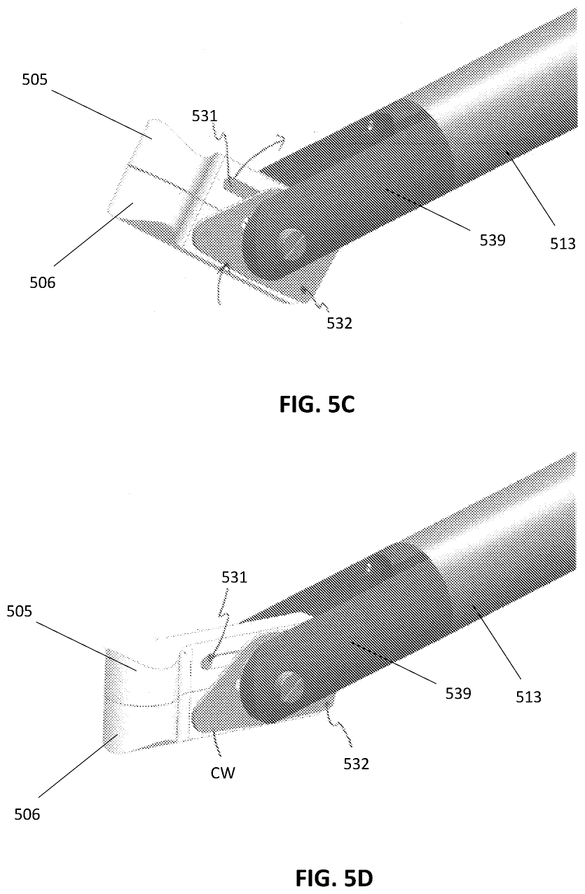

FIGS. 5A-5D illustrate another example of an elongate applicator tool as described herein. The elongate applicator tool of FIGS. 5A-5D may be configured with a rigid shaft to be used as a laparoscopic instrument. In this embodiment, the elongate applicator tool may be inserted through a canula. In addition, the elongate applicator tool of FIGS. 5A-5D may be configured with a flexible shaft to be used as an endoscopic instrument or catheter. In such embodiments, the elongate applicator tool may be inserted through the working channel of an endoscope or catheter sheath. As shown in FIG. 5A, the elongate applicator tool may include a pair of jaws (upper 505, lower 506) whose opening and closing can be controlled from the proximal end of the elongate applicator tool (not shown). In some examples, the opening and closing of the jaws may be controlled through a control (e.g., knob, etc.) located on or near a handle coupled to, or part of, the elongate applicator tool. In some other examples, the opening and closing of the jaws may be controlled directly or indirectly by a processor, state machine, embedded controller, or any other component, such as controller, including one or more processors. FIG. 5A shows the jaws open, while FIG. 5B shows the jaws closed. In this example, the jaws may form the articulating distal end region of the elongate applicator tool and may be configured (as shown and described in FIGS. 5C and 5D) to articulate by rotating up or down. The rotation of the jaws may be independent of the opening and closing of the jaws. In general, one or more electrodes may be coupled with the jaws and/or the distal end of the shaft 513 (the portion of the elongate applicator tool proximal to the articulating distal end region). In some examples the jaws may contain or themselves form the electrodes (e.g., plate or knife electrodes). For example, in FIGS. 5A-5D the upper jaw 505 may include the first electrode (e.g., a disc or plate electrode positioned on a first face 588 of the upper jaw 505), with a second electrode or electrodes on the opposite face of the lower jaw 506. In some examples the jaws may include one or more electrodes on or forming a portion of the jaws. As mentioned, the electrodes may be non-penetrative (as shown in FIGS. 5A-5B) or penetrative, such as needle electrodes, etc. In some examples non-penetrative disc or plate electrodes may contact and/or touch patient tissue without tissue penetration. For example, disc or plate electrodes may contact targeted tissue to apply pulse electrical fields (e.g., receive and apply a high voltage, pulsed energy treatment). As mentioned, in some examples the upper jaw 505 and the lower jaw 506 may include penetrative electrodes, such ss needle electrodes or the like (not shown), that penetrate the patient's tissue and deliver pulsed electrical fields (e.g., high voltage, sub-microsecond pulses).

FIGS. 5A-5D shows an elongate applicator tool including a jaw actuating mechanism that is configured to allow movement of the upper 505 and lower 506 jaws relative to each other (either both jaws may be moved to open/close as shown in FIGS. 5A-5B, or one jaw may move relative to the other, stationary, jaw) in an opening and closing action or motion. In FIGS. 5A-5B a pair of eccentric rotatable tabs 529, 530 with side pins 531, 532 are configured to be rotated in opposite directions (e.g., clockwise, CW, and counterclockwise, CCW) to cause the jaws move relative to each other, as shown in FIGS. 5A-5B. In this example, the jaws are coupled to both eccentric rotatable tabs 529, 530 with side pins 531, 532 with one slot in each jaw forming a slider opening(s) (or slot(s)) 537, 538. This configuration allows the jaws to close in parallel as the side pins 531, 532 of the eccentric tabs 529, 530 slide in the slider openings 537, 538 as they are articulated by rotating one or both eccentric rotatable tabs with side pins. The eccentric rotatable tabs with side pins may be actuated, for example, by a single member extending proximally (e.g., cable, rod, etc.). In some examples, there may be a linkage between two pulleys, the jaws and the distal end region 539, such that pulling or pushing on just one pulley may operate both pulleys to open/close the jaws. In FIGS. 5A-5B, to transition between open (FIG. 5A) and closed (FIG. 5B) jaws, the first distal eccentric rotatable tab 529 with side pin 531 is rotated clockwise (CW) while the second distal eccentric rotatable tab 530 with side pin 532 is rotated counterclockwise (CCW). These rotations are reversed to transition between the closed and open configurations.

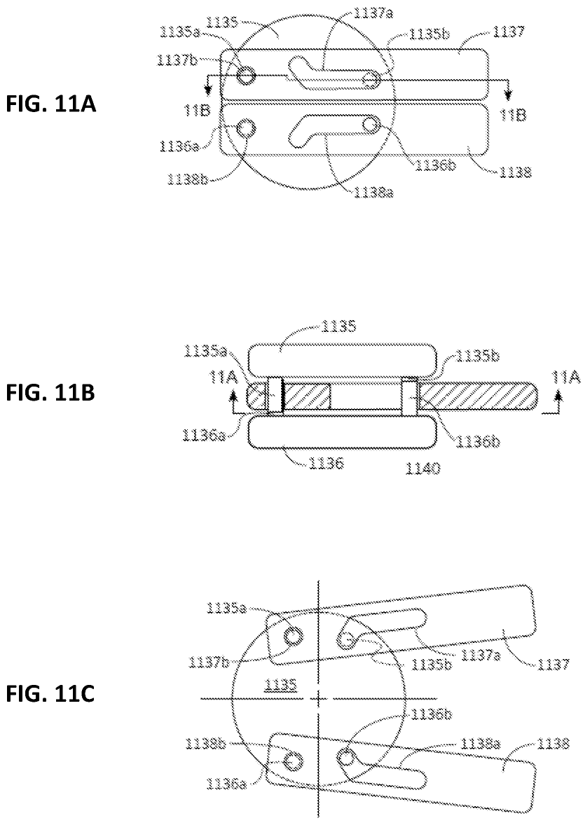

FIGS. 5A-5B illustrate articulation of the distal end region of the elongate applicator tool, including the upper and lower jaws 505, 506. The upper and lower jaws may be moved together, e.g., to rotate or bend relative to the distal end of the elongate applicator tool. In FIG. 5C, the jaws are shown rotated (angled) up relative to the elongate body of the elongate applicator tool (shaft 513). The jaws may be rotated up or down to angle the jaws up, as shown in FIG. 5C, and down or towards a neutral (e.g., straight-in line with the shaft 513) configuration, as shown in FIG. 5D. In various implementations, a separate elongate articulating member (e.g., a distal end region articulating member, such as a cable, rod, tendon, wire, etc.) may be part of an assembly for actuating the jaws of the distal end region. A jaw articulating pulley may be coupled to the one or more pulleys (e.g., eccentric rotatable tabs with side pins) used to open/close the jaws and may be configured to function independently or in coordination with the jaw opening/closing pulley. Some examples of such implementations are illustrated in FIGS. 11A-11C, 12A-12D, 13A-13C, 14A-14G.

FIG. 6 illustrates one example of a handle 600 of an elongate applicator tool as described herein. The elongate applicator tool of FIG. 6 may be configured with a rigid shaft to be used as a laparoscopic instrument. In this embodiment, the elongate applicator tool may be inserted through a canula. In addition, the elongate applicator tool of FIG. 6 may be configured with a flexible shaft to be used as an endoscopic instrument or catheter. In such embodiments, the elongate applicator tool may be inserted through the working channel of an endoscope or catheter sheath. The handle 600 may be configured to be hand-held (e.g., gripped) by a user for manual or semi-manual operation. The handle 600 is coupled to the elongate shaft 613 extending distally. A proximal portion of the handle 600 includes a housing 609; in FIG. 6 a portion of the handle housing 609 is formed into a finger grip 611. One or more controls (e.g., control elements or mechanisms) may be included as part of the handle 600 (or adjacent to the handle) to articulate the distal end region or tip of the elongate applicator tool, including opening/closing the jaws, rotating/bending the distal end region including the jaws, and in some examples (not shown) actuating the delivery of the high voltage pulsing. The handle 600 may include one or more controls to deploy and/or position electrodes located in or near the distal end region. The electrodes may include non-penetrating electrodes (e.g., surface, plate, disc electrodes, or the like) and penetrating electrodes (e.g., needle electrodes, or the like). In some examples, the control for actuating the high voltage, pulsing is separate from the handle 600 or is used in conjunction with a separate control, e.g., on the pulse generator or coupled to the pulse generator, e.g., a foot petal, switch, button, etc.

In FIG. 6, the apparatus includes a hinged finger control 622 that is pinned to the handle housing 609 to cause the jaws to open/close. Moving the finger control 622 relative to the housing (e.g., relative to the finger grip 611) drives the movement of the linkages connected to the jaws. The finger control 622 (which may be generically referred to a jaw open/close control) may be coupled to the jaw open/close elongate member by an assembly including one or more proximal jaw open/close eccentric rotatable tabs with side pins 641, 642, one or more drive pins 645, lever arms 646, etc. In the example shown in FIG. 6, the jaws may be closed by rotating the pulleys (e.g., eccentric rotatable tabs with side pins 642, 641) counterclockwise by moving the jaw control (finger control 622).

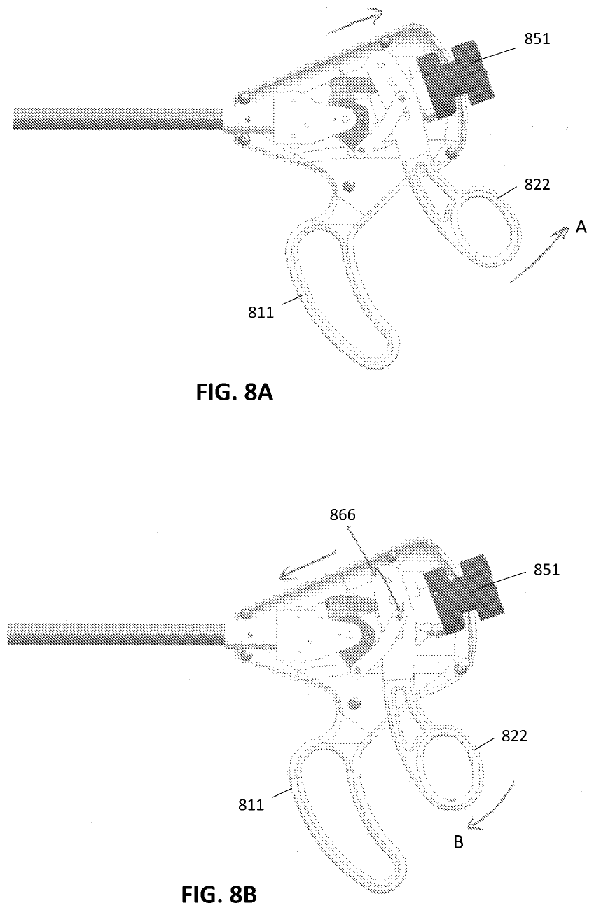

The distal end region of the elongate applicator tool may also be articulated to bend up or down (or in some examples, right or left) using a separate control or controls. In FIG. 6A an articulation control 651 (e.g., knob) may be coupled with an articulation assembly, including, for example, an articulation drive 653 for rotating the jaws as mentioned above. In some examples, as shown in FIG. 6 and described in detail in reference to FIGS. 8A-8B, the movement of the jaws may be limited, e.g., to limit how close together (or how far apart) the jaws may be moved. A control on the actuator may be used to limit the jaw opening and/or closing size. In FIG. 6 the articulation control 651 may also be configured or be part of the mechanism to adjust or limit the jaw opening/closing.

Any of the elongate applicator tools described herein may also be configured to be coupled to a pulse generator for delivering high voltage, sub-nanosecond pulses into the target tissue. In particular, the elongate applicator tools (apparatuses, devices, or systems including them) may be configured to isolate the high voltage power applied by the pulse generator from the hands of the operator to prevent accidental harm to the operator. In some examples, as shown in FIG. 6, the connection to the pulse generator may be an electrical cable 661 that is connected to the elongate applicator tool distally from the handle. In some examples the electrical cable connection to the pulse generator may be connected to the handle or proximally to the handle. In some examples it may be beneficial to connect distally of the handle to further isolate the operator from the high voltage energy applied by the pulse generator. The electrical cable 661 may be electrically isolated (and insulated) from the handle, and therefore the operator's hand(s), by one or more isolation elements. In some examples, in which the handle includes one or more electrical components, such as switches (including on/off switches communicating with the pulse generator), sensors (e.g., for sensing jaw opening distances, etc.) or the like, may be separately powered, using an electrical connection or method that is entirely isolated and/or separate from the electrical supply of the high voltage sub-microsecond pulsed energy applied to the tissue.

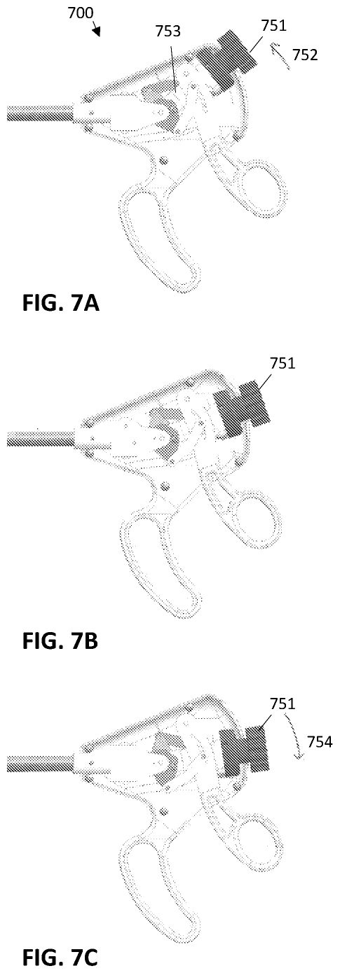

FIGS. 7A-7C illustrate one example of the operation of a proximal handle 700 similar to the one shown in FIG. 6 to adjust the angle of the distal end region (e.g., jaws) as shown in FIGS. 5C and 5D. In some examples, the jaws may include penetrating and/or non-penetrating electrodes to deliver pulsed electrical fields (e.g., high voltage, microsecond or sub-microsecond energy pulses). In this example, the jaws may be deflected down by moving the articulation control 751 up, in the direction shown by arrow 752 as shown in FIG. 7A. The linkage between the articulation control 751 and the articulation drive 753 within the handle, as well as one or more articulation members (e.g., bars, rods, wires, etc.) extending through the elongate shaft 613 (FIG. 6) of the elongate applicator tool forms an articulation assembly for adjusting the angle and spacing of the jaws. In some examples the control and/or articulation assembly may be configured so that moving the control down (rather than up), or rotating it (e.g., CW, CCW), moves the jaws down.

FIG. 7B shows the articulation control 751, and therefore the jaw rotation, in the center, neutral position (e.g., with the jaws straight, relative to the elongate shaft of the elongate applicator tool). FIG. 7C shows the articulation control 751 moved down in the direction shown by arrow 754, causing the jaws to rotate up at the distal end of the elongate applicator tool.

As mentioned above, the handle may include one or more limiters for limiting movement of the jaws and/or electrodes of the elongate applicator tool. In FIGS. 8A-8B the handle includes a jaw closure limiter that limits the movement of the jaws in closing, to prevent the jaws from completely closing (which may damage the tissue). For example, in FIG. 8A, the articulation control 851 is adapted to include a jaw closure limiter by rotating the dial (e.g., twisting it CW or CCW) to set the jaw closure limit. In FIG. 8A, the movement of the hinged finger control (e.g., control lever or control arm) 822 (rotating it CCW) moves the jaws apart, opening them as shown by arrow A.

In general, any of the methods and apparatuses described herein may control the field strength of the applied pulsed electric field by determining or confirming the spacing between the electrodes (as mentioned above) and estimating the field strength based on the spacing. As mentioned, any of these apparatuses may be configured to detect and/or indicate the spacing between the electrodes or sets of electrodes. For example, any of these apparatuses may be configured to output the spacing (e.g., by measuring the distance and/or angle directly or indirectly, including using a sensor to determine the distance. In some examples the apparatus and/or method of using an apparatus as described herein may adjust the applied energy to achieve a target pulsed field strength based on the determine distance between the electrodes. In some examples the apparatus may adjust the spacing or distance between the electrodes to achieve a target field strength (e.g., volts/cm, volts/in, or the like) associated with an applied energy (e.g., high voltage, microsecond or sub-microsecond energy pulses) that may be delivered to the patient. The spacing between electrodes or groups of electrodes may be directly related to the jaw closure amount; in some cases the maximum jaw closure may be the limit on the gap or spacing between the electrodes. In some examples, a user (e.g., doctor, nurse, or other clinician) may adjust a control (e.g., an articulation control 851) to determine the gap or spacing between the electrodes. In some cases, the articulation control 851 may be controlled directly or indirectly by a processor, embedded controller, stepper motor or the like. Thus, in some examples, the gap, and therefore the field strength, may be controlled by the apparatus (e.g., system 100). In some cases the spacing may be detected by the apparatus and used to estimate the applied energy. Thus, the apparatus may set the spacing to achieve a target electrical field strength and/or may adjust the applied energy using the detected or determined spacing.

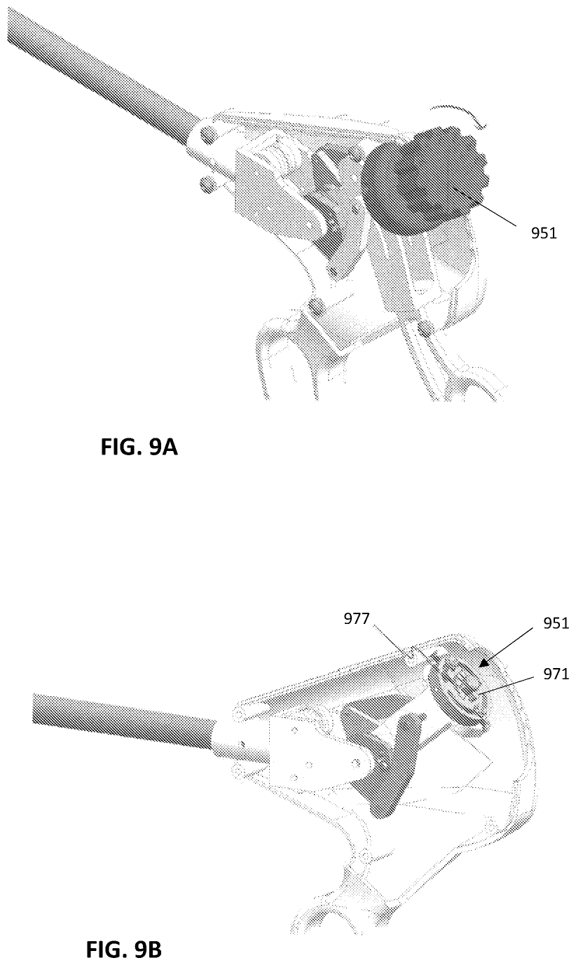

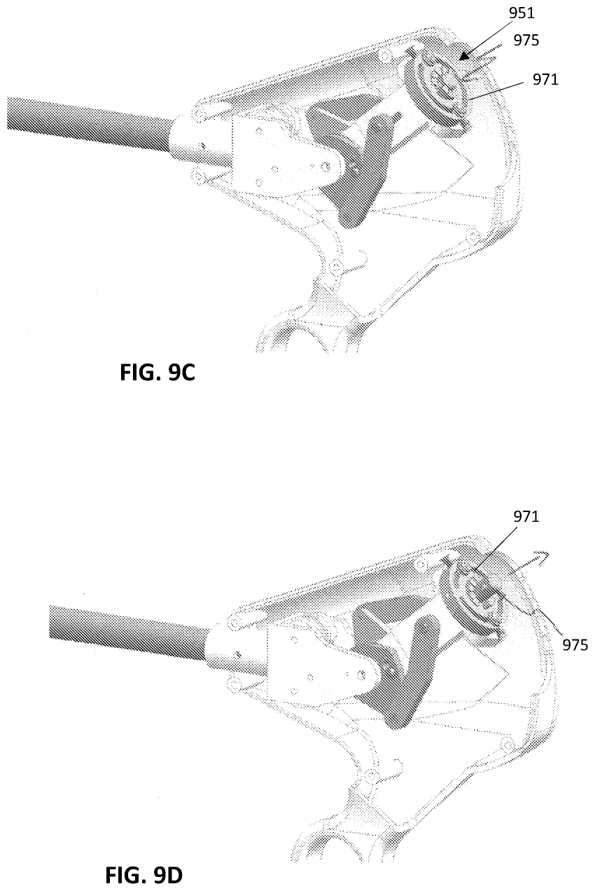

In some examples this movement may be transduced in part by a pin or rod that is pulled or pushed laterally (proximally or distally) as the jaws are opened or closed. As shown in FIG. 8B, the movement of the hinged finger control 822 in the opposite direction (e.g., rotating it CW) closes the jaws as shown by arrow B. In this example the jaw actuation pin 866 moves, driving an actuator forward to move the jaws to close at the distal end (not shown). This movement may be limited by adjusting the jaw limiter control, which may be part of the articulation control 851 which is configured as a dial, shown in FIG. 9A in greater detail.

In FIG. 9A, rotating dial knob of the articulation control 951 sets the jaw closure distance limit for the jaws. FIG. 9B shows the articulation control 951 with the dial knob removed. A circular spacer plate 971 within the articulation control 951 includes cam steps 977 that allow incremented selectable step adjustments to limit the jaw closure distance when selected. In some examples the control may be configured to permit continuous (non-step) adjustment of the jaw closure limit. The dial may adjust the position of the rod or pin forming part of the jaw closure actuation mechanism described above. The position of the circular spacer plate 971 and actuation rod 975 shown in FIG. 9C corresponds to condition when the jaws are fully closed, with the control (e.g., dial) set to a position where the motion of the actuation rod 975 is not impeded and does not prevent the jaws from fully closing. In the position of the circular spacer plate 971 and actuation rod 975 shown in FIG. 9D, the jaws are fully open (not shown).

Any of the elongate applicator tools described herein may include one or more controls for controlling extension/retraction of the one or more electrodes, including needle electrodes. In some examples an electrode control may be included on the handle (e.g., as a button, slider, knob, etc.) on the handle housing, and/or a trigger, e.g., on the finger grip region, or the like. The electrode control may be mechanical, as described above, including a mechanism, linkage, or the like for transmitting movement of the control to extend or retract the electrodes to one or more predetermined or continuously selectable positions (e.g., lengths). The needles may be fully retracted into the elongate applicator tool (e.g., the distal end of the elongate applicator tool) and/or they may be partially extended even when fully retracted. In some examples, the electrode control may be automated and include a stepper motor, servo, or the like to control the extension/retraction of the electrodes. As mentioned above, the electrodes may be surface, plate, disc, or other non-penetrating electrodes. The electrode control may determine a distance between the electrodes (including penetrative and non-penetrative electrodes).

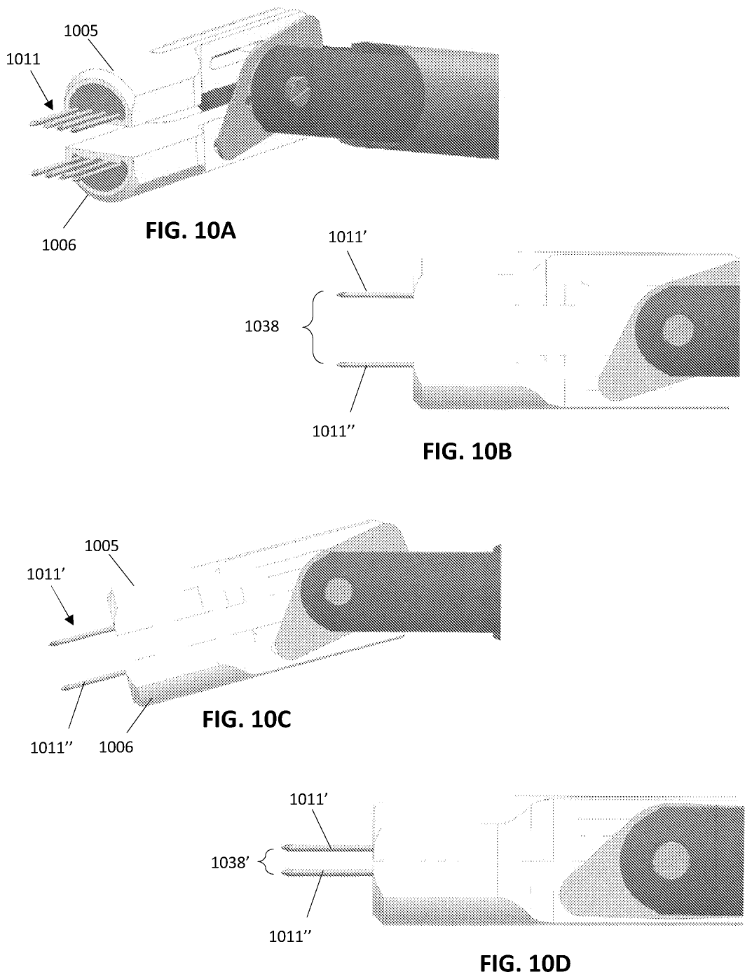

FIGS. 10A-10D illustrate another example of a distal end region of an elongate applicator tool as described herein. The elongate applicator tool of FIGS. 10A-10D may be configured as a laparoscope or an endoscope. The elongate applicator tool of FIGS. 10A-10D may be configured with a rigid shaft to be used as a laparoscopic instrument. In this embodiment, the elongate applicator tool may be inserted through a canula. In addition, the elongate applicator tool of FIGS. 10A-10D may be configured with a flexible shaft to be used as an endoscopic instrument or catheter. In such embodiments, the elongate applicator tool may be inserted through the working channel of an endoscope or catheter sheath. In FIG. 10A the elongate applicator tool includes jaws 1005, 1006 and a plurality of electrodes (configured as needle electrodes 1011) extending distally from the jaws and in a direction that is parallel to the plane of the jaws. Thus, opening and closing the jaws may adjust the distance (separation) between the electrodes; in some examples the electrodes may be configured as anode and cathode electrodes, such as anode on the upper jaw, cathode on the lower jaw, etc. (or vice versa) so that electric fields may be applied between them. FIG. 10B shows a side view of the distal end of the elongate applicator tool of FIG. 10A, showing the separation distance 1038 between the electrodes. In some examples all of the upper jaw electrodes 1011′ may be electrically connected (e.g., as anode) and all of the lower jaw electrodes 1011″ may be electrically connected (e.g., as cathode). FIG. 10C shows a side view with the jaws rotated as described above.

In FIG. 10D, the jaws have been substantially or completely closed, as discussed above. In some configurations the jaw travel may be limited to prevent complete closure. FIG. 10D shows a reduced separation distance 1038′ between the upper and lower electrodes. In various examples, the operator may set the distance (spacing) between the electrodes (or groups/rows of electrodes).

Although the example shown in FIGS. 10A-10D shows penetrative (e.g., needle electrodes 1011), in any of the apparatuses shown herein, including the example shown in FIGS. 10A-10D, the electrodes may instead be non-penetrative electrodes. For example, the distal end region of the elongate applicator tool of FIGS. 10A-10D may include surface, plate, disc, or other non-penetrating electrodes (not shown). In either case, changing the separation distance 1038, 1038′ between the jaws and therefore between the electrodes (or sets of electrodes) may affect a field strength of the high voltage pulsed energy treatment provided by the needle electrodes 1011 and/or the non-penetrating electrodes. As mentioned, the field strength may be adjusted based on the detected/determined spacing and/or the spacing may be adjusted to achieve a target fields strength.

As mentioned above, FIGS. 11A-11C illustrate one example of a mechanism for articulating the jaws (e.g., parallel jaws) 1137, 1138 to open and close. FIG. 11A shows a cut away view, showing one pulley 1135, in the assembled configuration, cut at cut line 11A-11A shown in FIG. 11B. The jaw opening/closing actuation assembly may include one more (in this example, two pulleys 1135, 1136) and one or more (in this example, four pins 1135a, 1135b, 1136a, 1136b that may fit within slots 1137a, 1138a and pin receiving holes 1137b, 1138b within jaw 1137, 1138. In FIG. 11A the jaws 1137, 1138 are shown in the closed configuration with respect to the pulley 1135, (pulley 1136 not visible in this cut away view). FIG. 11B shows a top cut-away view through the cut at 11B-11B shown in FIG. 11A. The pins 1135a, 1135b, 1136b, 1136a of the pulleys 1135, 1136 are positioned in holes 1137b, 1138b and slots 1137a, 1138a on each respective jaw 1137, 1138, so that rotating of first and second pulleys 1135, 1136 with respect to one another results in moving the jaws 1137, 1138 to the open configuration shown in FIG. 11C.

In the example of FIG. 11A, the jaw 1137 includes a slot 1137a that has a “dog leg” shape, having an angled section. The angled section allows non-parallel opening of the jaws at the end of travel, while the horizontal section of the slot allows parallel opening/closing. The slot may engage a pin from a first pulley, while the pin receiving hole (opening) 1137b engages a pin from a second pulley, as described above.

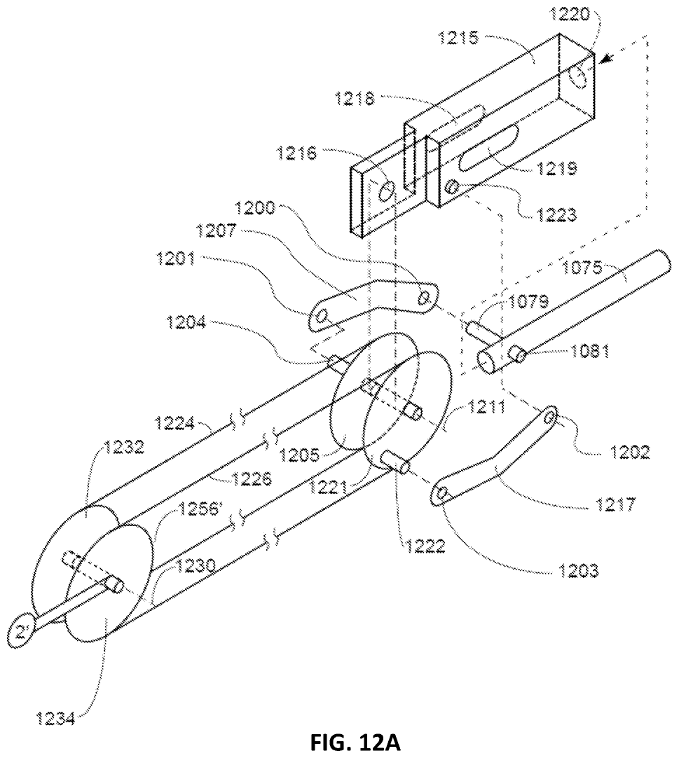

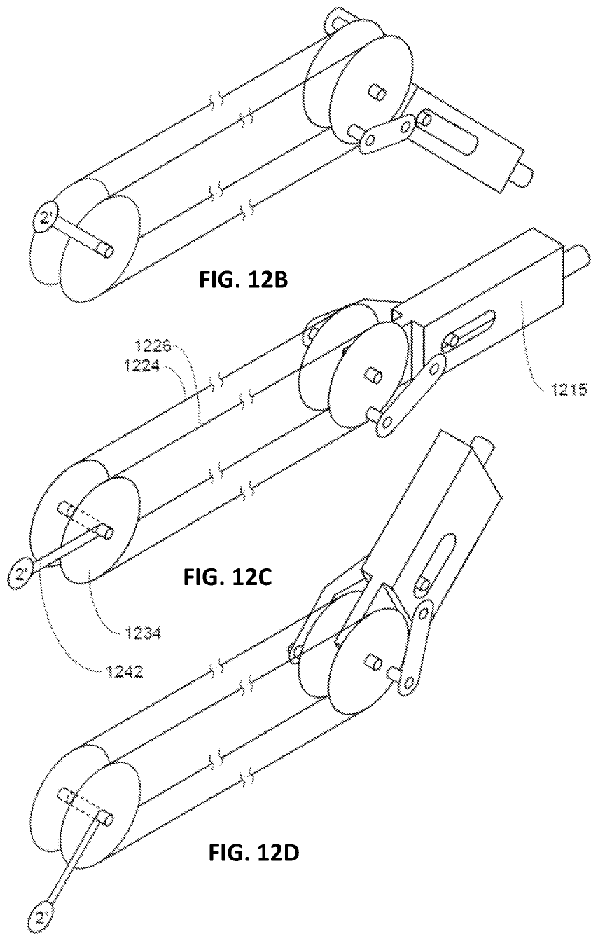

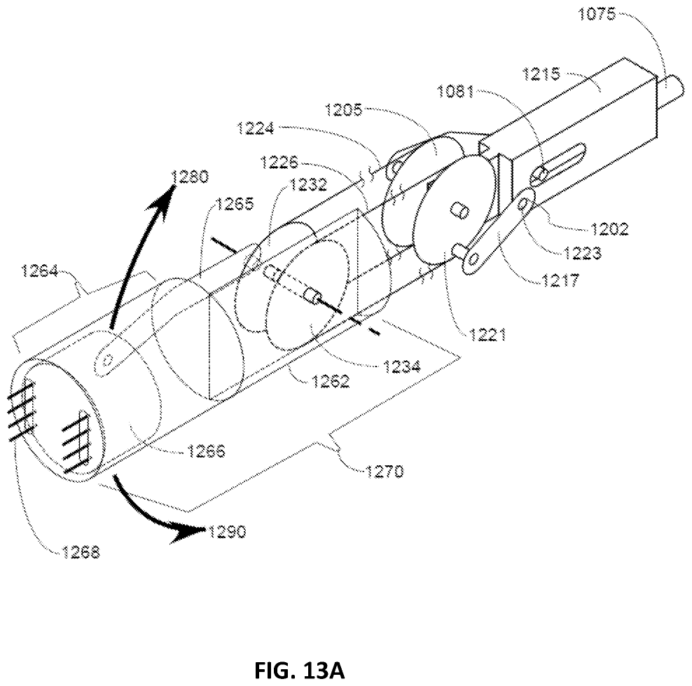

As previously stated, the elongate applicator tool of the present disclosure includes a proximal handle and a distal end region (treatment tip). The handle and the tip are operatively connected through an elongated (e.g., tubular) shaft, such as shaft 613 of FIG. 6. FIG. 12A is a perspective exploded schematic diagram showing an example of one example of the operative elements of the handle of the elongate applicator tool, which may be connected to the corresponding operative elements at the distal end region of the elongate applicator tool. FIG. 12A shows the assembly and/or connection paths for and between the various pieces. A rotation control housing 1215 includes a guide hole 1220 at the unconstrained proximal end of the housing 1215. The housing 1215 has first and second side slots 1218, 1219. A constrained (though rotatable) end of the housing 1215 includes a pivot hole 1216 through which the shaft 1211 of the drive pulleys, i.e., a first drive pulley 1205 and a second drive pulley 1221, passes. A rod 1075 is disposed in the housing 1215 through the guide hole 1220. A first rod side pin 1079 of the rod 1075 extends through the first side slot 1218 when the rod 1075 is disposed within the housing 1215 to engage a first hole 1200 in a first drive link 1207. A second rod side pin 1081 on an opposite side of the rod 1075 extends to a position within the second side slot 1219 when the rod 1075 is disposed within the housing 1215 to prevent the rod 1075 from twisting as it is moved axially within the housing 1215. A housing side pin 1223 engages a hole 1202 in a second drive link 1217. The first rod side pin 1079 and the second rod side pin 1081 may be a single unitary structure or may be separate from one another.

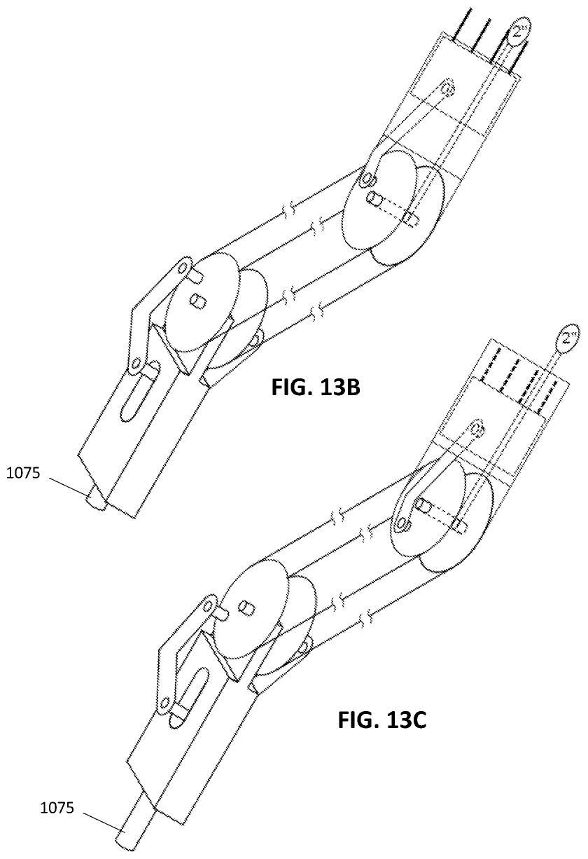

The first drive pulley 1205 and the second drive pulley 1221 may separately drive different portions of the distal end region of the device, of they may be coupled together to jointly drive different portions. For example in FIGS. 12A-12C (as will be illustrated in FIGS. 13A-13C) the second drive pulley 1221 drives rotation of the second slave pulley 1234, which may be coupled to a distal housing (e.g. holding electrodes, imaging elements, etc.), such as the electrode housing shown in FIG. 13A. The first drive pulley 1205 drives rotation of the first slave pulley 1232, which may be coupled to a second portion or element of the distal end region (e.g., housing), for example, to control extension or retraction of the housing covering the electrodes as illustrated in FIGS. 13B-13C.

A first connector 1224 (e.g., cable, rod, cord, etc.) is configured to be operatively connected between the first drive pulley 1205 and the first slave pulley 1232 such that a rotation of the first drive pulley 1205 causes a simultaneous rotation of the first slave pulley 1232 by a matching angle. A second connector 1226 (e.g., cable, rod, cord, etc.) is configured to be operatively connected between the second drive pulley 1221 and the second slave pulley 1234 such that a rotation of the second drive pulley 1221 causes a simultaneous rotation of the second slave pulley 1234 by a matching rotational angle. The drive pulley and the slave pulley will rotate through the same angle when the radiuses of the two match. The device can be configured with non-matching radiuses between the drive and slave pulleys to amplify or diminish the degree to which the slave pulley rotates when the drive pulley is rotated a particular angle. The direction of rotation may be controlled by the connection (connector 1224 or 1226) between the pulleys; the device can be configured so that the connectors cross (e.g., connect from the top of the drive pulley to the bottom of the slave pulley) so that the slave pulley rotates in the opposite (rather than the same) direction when the drive pulley is rotated. In the example of FIGS. 12A-12D, the connectors are arranged so that the drive pulley and the slave pulley rotate in the same direction (e.g., the top of the drive pulley is connected by the connector to the top of the slave pulley).

The drive pulleys rotate about a shaft 1211 a fixed distance from a slave pulley shaft 1230, about which the slave pulleys 1232 and 1234 rotate by a shaft of the elongate applicator tool (not show here). The fixed distance maintains constant tension in the drive to slave pulley connectors, such as connectors 1224, 1226, so that rotation of a drive pulley causes a similar rotation and motion at the distal end region of the elongate applicator tool.

Each drive pulley, such as 1205, 1221, may include an off center first drive pin 1204 and second drive pin 1222. The first drive pin 1204 is rotationally coupled with a receiving hole 1201 of the first drive link 1207. The second drive pin 1222 is rotationally coupled by with a receiving hole 1203 of the second drive link 1217.

FIGS. 12B, 12C and 12D show relative motion of the housing 1215 at the proximal handle of the elongate applicator tool of the present disclosure as such motion relates to motion (rotation) of the slave pulleys at the distal end region of the elongate applicator tool. The distal end region of the device may be moved by the second drive pulley 1221 acting on the second slave pulley 1234, to which the distal end (e.g., shown as a second position indicator 1242 with the distal marker 2′ in FIGS. 12A-12D) is coupled. In the schematic diagrams of FIGS. 12B-12D the second position indicator 1242 (2′) is shown coupled to the second slave pulley 1234; this position indicator 1242 illustrates the relative rotational position of the second slave pulley 1234 as it tracks the rotation of the housing 1215 so that the angular rotation between the two ends (the housing 1215 end and the slave pulleys end) is matched.

FIG. 12C illustrates the housing 1215 oriented in-line with the axis of the connectors 1224, 1226. The rotational orientation of the first and second slave pulleys is indicated by their respective position indicators (only indicator 1242 is shown), and they are also oriented in-line with the axis of the elongated shaft of the elongate applicator tool (not shown). In practice, the angular orientation of the respective ends can be in any rotational direction with respect to the other, but the rotational motion of the proximal drive end will cause a corresponding relative rotation at the distal end region. As can be observed in FIG. 12B, when the housing 1215 is rotated to a “down” position as shown, both slave pulleys 1232, 1234 are rotated to an “up” position as indicated by the upward rotation of position indicators, such as 1242. As can be observed in illustration of FIG. 12D, when the housing is rotated to an “up” position as shown, both slave pulleys 1232, 1234 are rotated to a “down” position as indicated by the downward rotation of the position indicator 1242.

FIG. 13A is a schematic representation of the elongate applicator tool as shown in FIG. 12A with additional details of the distal end region of the elongate applicator tool shown (e.g., as a tip including electrodes) to illustrate the mechanisms causing the rotation of the electrode housing 1264 at the distal end region of the elongate applicator tool. As illustrated above, the position (angle 1280) of the distal end region may be controlled by the second drive pulley 1221 and second slave pulley 1234. In addition, the other set of pulleys (the first drive pulley 1205 and the first slave pulley 1232) may control extension and retraction of a needle/electrode holder 1266 on or forming part of the electrode housing 1264 of the distal end region. Movement of the drive pulley 1205 and slave pulley 1232 (shown in FIG. 13A) pushing on the electrodes (shown as needle electrodes 1268) in and/out of the electrode housing 1264; alternatively a slidable electrode housing may be moved distally or proximally to cover or expose the electrodes. Thus, the housing, or the electrode base may be coupled to an extension/retraction link 1265 that connects to the first slave pulley.

In general, the rotation of the slave pulleys, such as pulleys 1232, 1234, may be directly linked through the connectors 1224, 1226, to the proximal end drive pulleys (e.g., 1205, 1221) so that motion of each drive pulley causes a corresponding motion of each respective connected slave pulley, as described above in FIGS. 12A-12D. In some examples, motion of the drive pulleys 1205 and 1221 may be controlled through stepper motors, rotary actuators, or the like under further control of a processor, state machine, embedded controller, or the like included in the system 100 (not shown). Thus, in some examples, penetration depth of the needle electrodes 1268 may be controlled, including adjusting up/down, to provide or maintain a desired energy field. Alternatively or additionally the pulse generator may be configured to adjust the output energy (e.g., voltage, current) based on the spacing between the electrodes or sets of electrodes.

FIGS. 13B and 13C are perspective back side view schematic diagrams of the example of the handle portion and distal end region assembly of the apparatus of the present disclosure, such as those shown in FIG. 12A. FIGS. 13A-13C show an attached distal end region (having a needle holder). The needle holder (and the optional position indicator labeled 2″ similar to the one described in FIG. 12A-12D) may be tilted by moving the handle of the device, as described above. The distal end region is coupled to the operative pivoting element at the handle (e.g., through the second drive pulley and the second slave pulley), while the first drive pulley and first slave pulley is coupled to the rod 1075 within the handle. In FIG. 13B the rod 1075 is advanced distally, causing the first drive pulley to rotate forward, which then causes the first slave pulley to rotate forward, to cause the needle holder to move forward to extend the needles from the electrode housing, as shown. Note that in this example, the needle holder may be moved separately from the articulation of the distal tip region. In FIG. 13C the rod 1075 is pulled back (proximally) from the housing, causing the first drive pulley to rotate back, which causes the first slave pulley to rotate back, causing the needle holder to move back and retract the needles into the electrode housing.

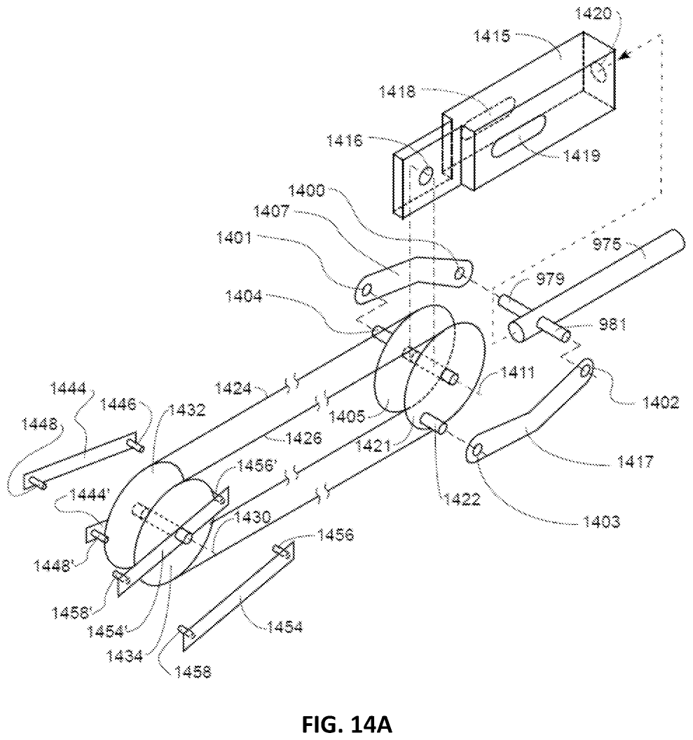

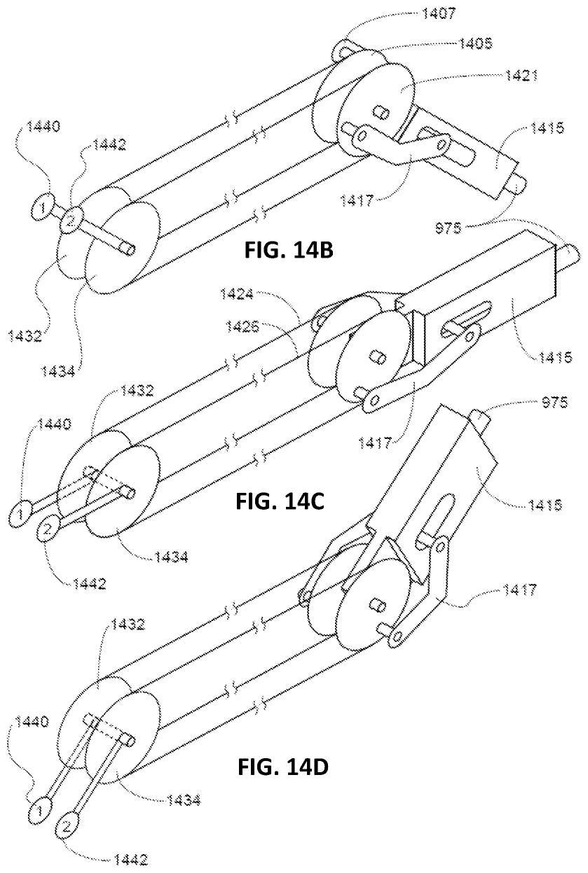

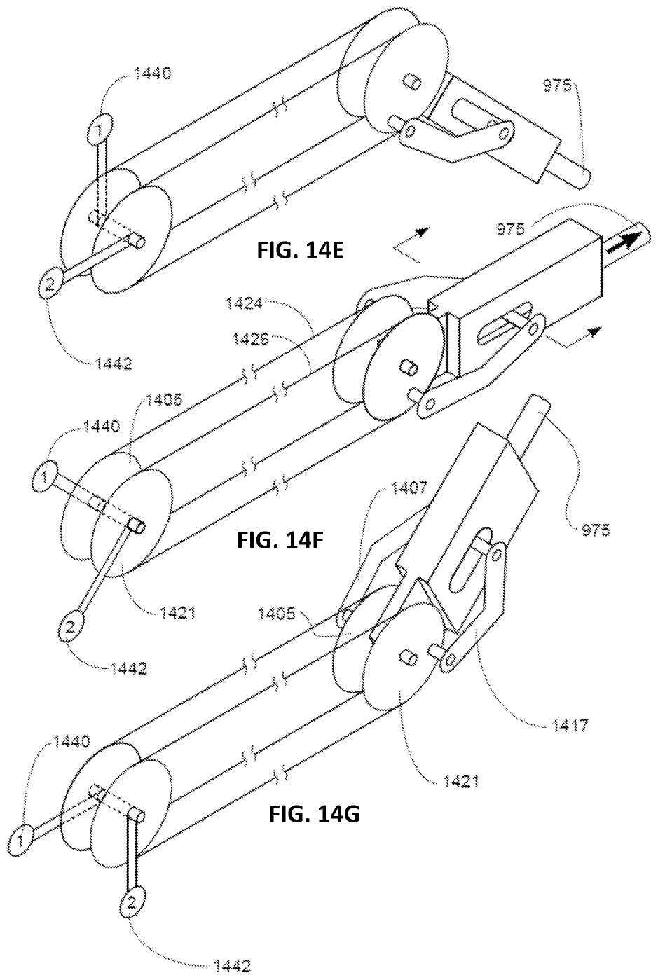

FIGS. 14A-G illustrate another example of the operative elements (including a handle) to control articulation of the distal end region of the elongate applicator tool of the present disclosure. In this example the two control sub-systems, e.g., the first drive pulley and the first slave pulley (that may control exposing the electrodes, as shown in FIGS. 13A-13C) and the second drive pulley and second slave pulley (that may control the articulation of the distal tip region, as shown in FIGS. 12A-12D) are coupled together so that they may be actuated together. For example, in FIG. 14A, the drive links 1407 and 1417 may be similar to those shown in FIG. 12A but may both be lined to and engage the actuation rod 975 within the housing 1415 in the handle. In this example, a first side pin 979 is fixed to a side of the activation rod 975 and, when the actuation rod 975 is disposed within the housing 1415, the first side pin 979 extends out through the first side slot 1418 to engage a first receiving hole 1400 in a distal end of first drive link 1407. A second side pin 981, fixed to an opposite side of the activation rod 975 (opposite from the first pin 979), extends from the actuation rod 975 and, when the actuation rod 975 is disposed within the housing 1415, the second side pin 981 extends out through the second side slot 1419 to engage a hole 1402 in one end of the second drive link 1417. The first side pin 979 and the second side pin 981 may be a single unitary structure or may be separate from one another as described herein.

A first connector 1424 (e.g., a pulley cable) which is operatively connected between the first drive pulley 1405 and the first slave pulley 1432, such that a rotation of the first drive pulley 1405 through a first rotational angle causes a simultaneous rotation of the first slave pulley 1432 through a matching rotational angle. A second connector 1426 (e.g., a second pulley cable) which operatively connected between the second drive pulley 1421 and the second slave pulley 1434, such that a rotation of the first drive pulley 1405 through a first rotational angle causes a simultaneous rotation of the first slave pulley 1432 through a matching rotational angle. The angular rotation of the drive pulley and the slave pulley will match when the radiuses of the first drive pulley and first slave pulley match, as described above, assuming the connector is sufficiently rigid. The system can be configured with non-matching radiuses to amplify or diminish the degree to which the slave pulley rotates when the drive pulley is rotated a particular angle. The system can be configured with the connectors 1424, 1426 connecting the top of one pulley to the bottom of the other pulley, so that the slave pulley rotates in the opposite (rather than the same) direction when the drive pulley is rotated.

The drive (control) end pulleys rotate about a drive end pulley center shaft/axis 1411 which is mounted within an operator's handle as described above, e.g., a fixed distance from a treatment end slave pulley center shaft/axis 1430 about which the slave (treatment) end pulleys rotate, e.g., by a shaft (not show). The fixed distance may maintain a constant tension in the drive to slave connectors (e.g., connectors 1424, 1426) so that rotation of a drive pulley at the drive end immediately and simultaneously causes a similar rotation and motion at the treatment end of the tool.

Each drive pulley 1405, 1421 includes an off center pin, i.e., first drive pin 1404 and second drive pin 1422. The first drive pin 1404 is rotationally coupled by a receiving hole 1401 to the first drive link 1407. The second drive pin 1422 is rotationally coupled by a second receiving hole 1403 to the second drive link 1417.

Each slave pulley 1432, 1434 is coupled to or configured as an integral unit with two eccentric jaw opening control drive pins. A set of first pins 1446, 1448 (shown mounted on a first mounting bar 1444) are shown separate from the first slave pulley 1432 for clarity of illustration: a mounting bar 1444 and the pins 1446, 1448 appear as eccentric mounting bar 1444′ and pins 1448′, 1446′ (not shown), when attached to or integral with the first slave pulley 1432. A set of second slave pulley drive pins 1456, 1458 (shown mounted on a second slave pulley drive pin mounting bar 1454), shown separate from the second slave pulley 1434 for clarity of illustration: the mounting bar 1454 and the pins 1456, 1458 appear as eccentric mounting bar 1454′ and pins 1456′, 1458′ when attached to or integral with the second slave pulley 1434. The mounting bar may mount an operative portion of the tip (e.g., the tip body, the electrode base, etc.) to the first slave pulley. As described above, the treatment end jaw movement may be controlled by the same or opposite rotational direction motion of the first slave pulley and its eccentric pins and the rotational motion of the first slave pulley.

For example, the rotation of the slave pulleys 1432, 1434 may be directly linked through a connector 1424, 1426 (e.g., cable, rod, etc.) to the drive pulleys 1405, 1421 so that motion (rotation) of each drive pulley causes a corresponding motion (rotation) of each respective connected slave pulley.

FIGS. 14B, 14C, and 14D show motion of the rotation control housing 1415 at the proximal end of the apparatus (e.g. catheter) as such motion relates to motion (rotation) of the slave pulleys at the distal end region of the catheter. In the schematic diagrams of FIGS. 14B, 14C, and 14D a first position indicator 1440 is shown fixed to the first slave pulley 1432 and illustrates the relative rotational position of the first slave pulley 1432, while a second position indicator 1442 is shown fixed to the second slave pulley 1434 and illustrates the relative rotational position of the second slave pulley 1434. As mentioned above, in operation, the distal end region operable components (e.g., electrode holder, slidable cover, etc.) may be coupled to the slave pulleys (first or second) to control operation of two different operable components of the tip.

FIG. 14C illustrates the control housing 1415 oriented in-line with the axis of the connectors 1424, 1426, where the rotational position orientation of the first and second slave pulleys is indicated by their position indicators, i.e., 1440, 1442 and, therefore, the elongate applicator body (not shown) is oriented in-line with the axis of the connectors. In practice, the angular orientation of the respective ends can be in any rotational direction with respect to the other, but the rotational motion of the drive end will cause a corresponding relative rotation at the distal end region. As can be observed in illustration of FIG. 14B, when the control housing 1415 is rotated to a “down” position as shown, both slave pulleys 1432, 1434 are rotated to an “up” position as indicated by the upward rotation of the position indicators 1440, 1442. As can be observed in illustration of FIG. 14D, when the control housing 1415 is rotated to an “up” position as shown, both slave pulleys 1432, 1434 are rotated to a “down” position as indicated by the downward rotation of the position indicators 1440, 1442.

FIGS. 14B, 14C, and 14D provide illustrations of where drive pulleys and the slave pulleys are in what might be considered an aligned orientation (which may match a treatment jaw closed configuration, for example), FIGS. 14E-14G illustrate an example of the effect of actuation rod (e.g., “jaw actuation rod”) 975 moving proximally such that the first side pin 979 and the second side pin 981 cause the first drive link 1407 and the second drive link 1417 to move proximally such that first pin 1404 and second pin 1422 are moved with their respective drive links in a proximal direction. The proximal movement of the drive pins 1404, 1422 causes the first drive pulley to rotate in a clock-wise direction as the first link 1407 is pulled proximally, while the second drive pulley rotates in a counter-clock-wise direction as the second link 1417 is pulled proximally. This counter rotational motion of the drive pulleys causes a corresponding counter rotational motion of the slave pulleys, so that the slave pulleys move approximately 90 degrees relative to one another, as can be observed by the position of the position indicators 1440, 1442 in FIGS. 14E-14G, offset from one another. FIGS. 14E, 14F, and 14G show the indicators (“1” and “2”) coupled to the first and second slave pulleys, respectively, separated from each other (e.g., in an “open” configuration) and rotated “up”, “centered”, or “down,” respectively, as the control housing is shown rotated down, centered, or up. FIG. 14F illustrates the operation of the first drive and first pulleys 1405, 1421 in their respective pulley systems that control opening of the jaws by pulling on connectors (e.g., connectors 1424, 1426). For example, to open a pair of jaws, each of the jaws may be independently coupled with the slave pulley and linked to the drive pulley via a connector and to the actuation rod 975. The rod 975 may be retracted (e.g. moved in a proximal direction) to cause the pulley systems to rotate with respect to one another to open the jaws. To close the jaws, the rod 975 may be pushed in to cause the pulley systems to rotate (in the opposite direction to opening) with respect to one another to close the jaws. FIG. 14F shows the operative elements in the handle connecting with the moving elements at the distal end region of the elongate applicator tool where the pivot elements are positioned in a generally linearly aligned configuration, while moving a rod 975 in a proximal direction (shown by an arrow) causes the rotating elements at the distal end region of the elongate applicator tool to rotate in opposite directions.

FIG. 14G shows operative elements in the handle connecting with the moving elements at the distal end region of the device (e.g., at the end of the elongate applicator body) where the control housing is pivoted up to cause the distal end moving elements (indicated by 1 and 2) to pivot down. FIG. 14E shows the operative elements in the handle connecting with the moving elements at the distal end region of the elongate applicator tool where the control housing is pivoted down to cause the distal end moving elements (indicated by 1 and 2) to pivot up.

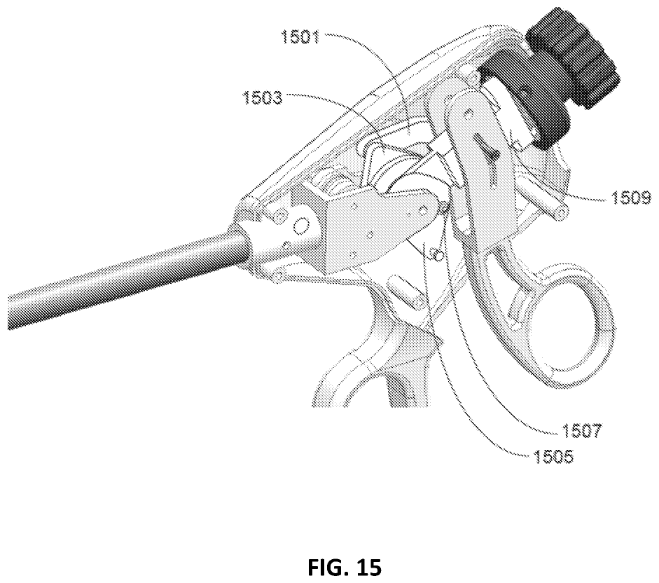

FIG. 15 shows a cut-away view of a proximal (handle) end of the elongate applicator system described in FIGS. 12A-12D. There is only one link 1501 connecting from the first pulley element 1503 to the articulating element 1509 to control the extension and retraction of the needle holder, while the second pulley element 1505 is fixed to the body of the articulating element 1509 by a pin 1507, thus causing the articulating element 1509 and the second pulley element 1505 to move (rotate) in unison.

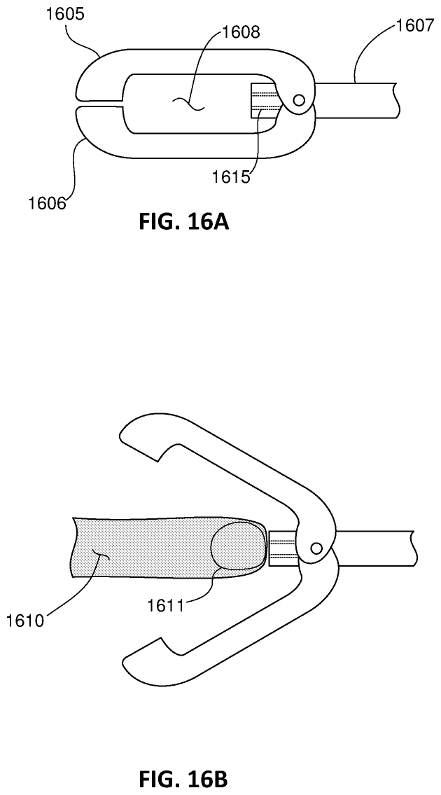

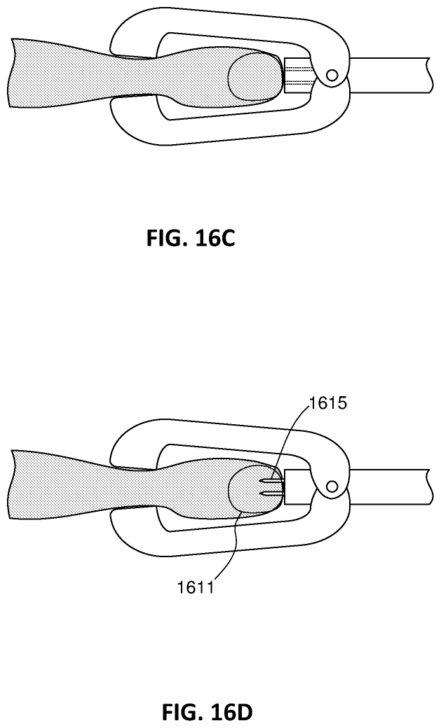

FIGS. 16A-16D illustrate another example of an elongate applicator tool as described herein in which the elongate applicator tool includes a set of grasping jaws providing a mechanism for securing tissue prior to electrode deployment, while the electrodes may be extended from the shaft of the elongate applicator tool into the tissue secured by the jaws. In general, the jaws may pivot on a pin, and there may be some offset to a slot or linkage that is driven by a push/pull rod that goes through the shaft back to the handle. The rod may connect to a thumb ring or lever actuated, for example, by fingers and the jaws may be locked closed. In some examples electrodes (e.g., needle electrodes) may extend from the shaft using a similar push/pull rod mechanism. This may also be driven by a thumb ring, finger lever, or slide. The two mechanisms can be independent so the user could clamp and then extend the needles into the stabilized tissue. In some examples, the handle may have a switch on it to activate pulse treatment or the foot pedal could be used. The elongate applicator tool of FIGS. 16A-16D may be configured with a rigid shaft to be used as a laparoscopic instrument. In this embodiment, the elongate applicator tool may be inserted through a canula. In addition, the elongate applicator tool of FIGS. 16A-16D may be configured with a flexible shaft to be used as an endoscopic instrument or catheter. In such embodiments, the elongate applicator tool may be inserted through the working channel of an endoscope or catheter sheath.

In FIG. 16A, an upper jaw 1605 and a lower jaw 1606 may be opened or closed in order to grasp tissue between the two 1608; the jaws are shown closed in FIG. 16A. The shaft of the elongate applicator tool 1607 houses one or more electrodes 1615 (e.g., needle electrodes) that may be extended distally into the tissue held between the upper and lower jaws. This is illustrated in FIGS. 16B-16D. In FIG. 16B tissue 1610 between the two jaws, including a target region of tissue 1611. The jaws, even when closed, in this example include a gap for holding the tissue and for the electrodes. In FIG. 16C the tissue and target region in particular is secured in the gap of the jaws, so that, as shown in FIG. 16D, the electrodes (e.g., needle electrodes 1615) may be extended out distally into the target region of the tissue. High voltage, sub-microsecond pulsed electrical energy may then be applied, as described above. In this example the jaws are configured as integrated grasper jaw. This jaw may be used to secure the target tissue and stabilize it before the needles are extended. This may aid in targeting as well as reduce tenting of the tissue (e.g., compressing the tissue in as the needle electrodes are inserted).

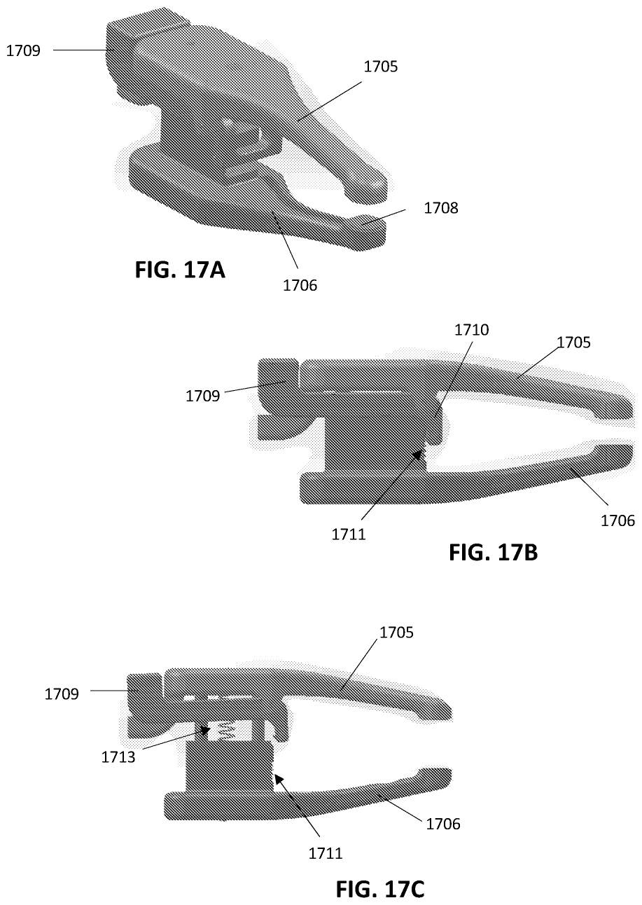

FIGS. 17A-17C illustrates another example of a distal end region of an elongate applicator tool as described herein, including a pair of jaws (e.g., upper 1705 and lower 1706 jaws) configured to close in parallel as part of a pinch clamp including a ratcheting tooth region 1711 on the lower jaw having teeth at intervals. The elongate applicator tool of FIGS. 17A-17C may be configured with a rigid shaft to be used as a laparoscopic instrument. In this embodiment, the elongate applicator tool may be inserted through a canula. In addition, the elongate applicator tool of FIGS. 17A-17C may be configured with a flexible shaft to be used as an endoscopic instrument or catheter. In such embodiments, the elongate applicator tool may be inserted through the working channel of an endoscope or catheter sheath. In practice, the user may close the ends of the device over a target tissue to achieve the desired clamp force. Distal ends of the upper 1705 and lower 1706 jaws may include plate electrodes 1708 (plate electrode 1708 on lower jaw 1706 is shown while the plate electrode disposed on upper jaw 1705 may be occluded from view). The distance between the plates may be detected or user determined (e.g., based on how far it is clamped) and/or a sensor may automatically provide the plate spacing to the generator. In this manner, the system 100 may change or control a voltage provided by the pulse generator to the plate electrodes 1708 to maintain a desired field strength.

In some examples, the distal end region may include a sensor or transducer that determines (e.g., measures) a distance between the upper 1705 and lower 1706 jaws. The distance may be provided to the system 100. The system 100 may, in turn, determine a voltage to provide by the pulse generator to the plate electrodes 1708 to provide a desired field strength based on the determined distance.

In general, any of the elongate applicator tools described herein may be configured to measure the jaw distance, and therefore (in some examples) the separation between the electrodes, e.g., anode(s) and cathode(s). Example electrodes may include plate electrodes 1708 or other non-penetrating electrodes as well as penetrating electrodes (not shown). Treatment efficacy may depend on delivering a specific field strength to the target tissue. The field strength is dependent on the spacing between the electrodes delivering the pulse, so knowing the spacing may be important for delivering a correct treatment. Any of the high voltage, sub-microsecond pulsing elongate applicator tools described herein may provide precise stops, preset distances, or sensor feedback to allow the operator, user, and/or system 100 to determine the width or spacing between electrodes. For example, the distal end region of FIGS. 17A-17C may include measurement markings (not shown) that indicate a distance between the upper 1705 and lower 1706 jaws (and therefore a distance between plate electrodes 1708. These measurements may be manually and/or automatically detected. For example, a user (e.g., a clinician) can observe the indicated distance and provide this distance to the system 100, for example through a user interface. Alternatively or additionally, the markings may be directly detected and provided to the system (e.g., the pulse generator). For example, a sensor may be used to detect the spacing between the jaws (and therefore the electrodes). In some examples the apparatus may include a sensor that determines (e.g., measures) a distance between the upper 1705 and lower 1706 jaws, e.g., at a distal end region.

The measured distance may be provided to the system 100. The system 100 may include a processor, embedded controller, or the like that controls the pulsed voltage and/or current provided to the electrodes. In some examples, the system 100 can vary the voltage and/or current based on the distance between the electrodes. For example, the system 100 may determine a voltage to provide from the pulse generator to the electrodes commensurate with the reported or determined distance. In this manner, the system 100 may provide and/or control a desired field strength with respect to the electrodes. Although described with respect to FIGS. 17A-17C, any of the apparatuses described herein may be configured to detect the spacing (e.g., gap, distance, etc.) between the electrodes or pairs or electrodes and/or to infer this distance from the spacing or distance of the jaws or one or more controllers controlling the spacing between the jaws. In any of these apparatuses, the control of the voltage and/or current supplied to the electrodes to maintain or control the field strength may be provided by the system 100 to any feasible electrode including the electrodes of FIGS. 3A-3B, 4A-4D, 5A-5D, 10A-10D, 16A-16D, 18A-18C, and 19A-19B.

Any of the apparatuses described herein may be configured to keep the electrodes (e.g., including plate electrodes, e.g., 1708, needle electrodes, etc.) parallel so that the field is consistently applied across the tissue.

FIGS. 17A-17C show one example of a distal end region of an elongate applicator tool configured as a parallel closing pinch clamp with ratchets set at known intervals. The operator may close the ends of the device over the target tissue to achieve the desired clamp force. Alternatively, this configuration may be used as a clamp attachment without being part of an elongate applicator tool.

As mentioned above, in any of these examples, the distance between the electrodes may be manually or automatically (or semi-automatically) determined and transmitted to the pulse generator. For example, a user (e.g., clinician) may input the distance between the plates, or a sensor or other transducer may automatically provide the jaw (and therefore the electrode, including plate electrodes, needle electrodes, etc.) spacing to the system, including the pulse generator. In FIGS. 17A-17C, the distal end region includes the upper jaw 1705 and the lower jaw 1706 and a ratcheting connection between the upper and lower jaw. The ratcheting connection may include a bias 1713 holding the jaws apart (or in some examples, holding them together) and a pawl 1710 and tooth region 1711 forming the ratchet region. The clamp structure shown in FIGS. 17A-17C may be coupled to an elongate shaft and proximal handle, as described above. An actuator (e.g., an elongate member such as a rod, wire, etc.) may be coupled to the ratchet release 1709 and may be used to apply force to close the upper and lower jaws when the ratchet is released.

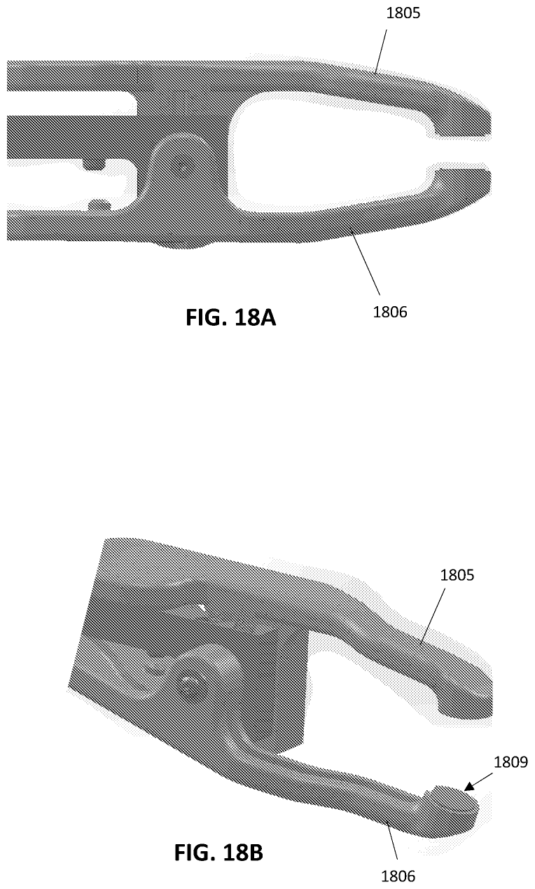

FIGS. 18A-18B illustrate another style of a clamping distal end region that may be integrated as part of an elongate applicator tool or used separately as a clamp to deliver high voltage, sub-microsecond pulses. The elongate applicator tool of FIGS. 18A-18B may be configured with a rigid shaft to be used as a laparoscopic instrument. In this embodiment, the elongate applicator tool may be inserted through a canula. In addition, the elongate applicator tool of FIGS. 18A-18B may be configured with a flexible shaft to be used as an endoscopic instrument or catheter. In such embodiments, the elongate applicator tool may be inserted through the working channel of an endoscope or catheter sheath. In FIGS. 18A-18B the distal end region is configured to include a pair of jaws 1805, 1806 that are attached to a pivot and include an opening that can be preset so that in the closed position the electrodes (e.g., plates) 1809 are a known distance apart.

In use, an operator may pre-select the setting (e.g., the distance separation setting), then apply the jaws to clamp to the target tissue. As described above, this configuration of the jaws may be part of a distal end region of the elongate applicator tool that has a set predetermined clamping distance, or it may be operated separately from an elongate applicator tool as a tissue clamp. In some examples, the system 100 may sense or otherwise determine a predetermined clamping distance of the distal end region of FIGS. 18A-18B, and therefore the spacing between the electrodes or sets of electrodes. The system 100 may determine a power (e.g., voltage) to be applied based a selected (manually or automatically by the system 100) field strength, which may be determined in part based on the distance between the electrodes. Thus the system 100 may provide a desired field strength for treatment. In some examples, all or some of the electrodes may be needle electrodes (or any other feasible penetrating electrodes) and may be disposed or mounted on the jaws 1805, 1806.

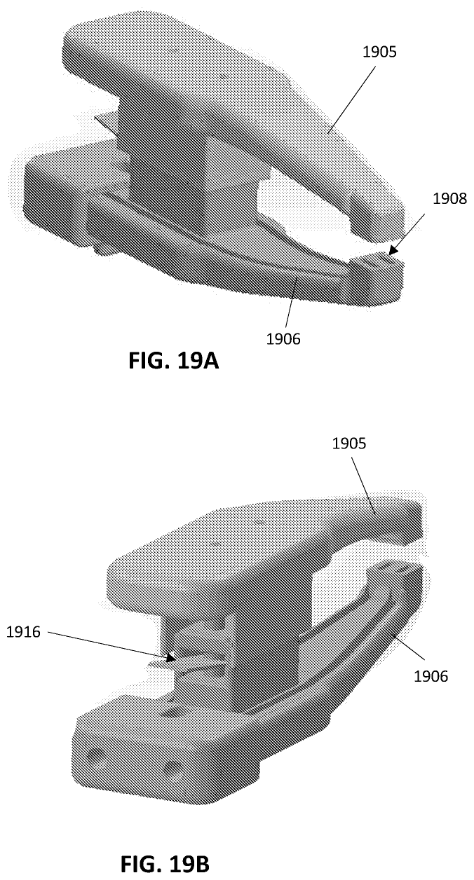

FIGS. 19A-19B illustrate another example of a distal end region of an elongate applicator tool (or alternatively, a clamp) that is configured to determine the spacing or distance between the upper and lower electrodes. The elongate applicator tool of FIGS. 19A-19B may be configured with a rigid shaft to be used as a laparoscopic instrument. In this embodiment, the elongate applicator tool may be inserted through a canula. In addition, the elongate applicator tool of FIGS. 19A-19B may be configured with a flexible shaft to be used as an endoscopic instrument or catheter. In such embodiments, the elongate applicator tool may be inserted through the working channel of an endoscope or catheter sheath. The upper jaw 1905 and lower jaw 1906 may be separated by an adjustable distance. In FIG. 19A, the jaws house one or more retractable needle electrodes. In some examples two or more rows of needle electrodes (not shown) are included and extend out of the same jaw (i.e. out of the slots 1908), or one row of needles may extend out of each jaw so that opposite polarities are in opposing jaws. With two jaws, the needle electrodes may have a back stop which prevents the needle electrodes from extending beyond the stop into undesired areas. In addition, providing a clamp force on the tissue to be treated positioned between the jaws may reduce arcing by reducing tenting of the tissue around the needles. For example, in FIG. 19A, the distal end region may be configured as a retractable needle style pinch electrode; in some examples, this distal end region is coupled to a shaft of the elongate applicator tool. The jaws may include two or more rows of needles (not shown) extending out of the same jaw or in some examples, one row of needles extending out of each jaw so that opposite polarities are in opposing jaws.

FIG. 19B is similar but is configured to be infinitely adjustable. The distal end region may also be configured to lock at a particular (e.g., selected) jaw distance. In practice, the upper jaw 1905 and the lower jaw 1906 may be adjustably set to a particular separation distance for the electrodes, so that the operator may squeeze the jaws together with a desired force and the jaw then stay the set distance apart when the user removes the squeezing clamping force. In FIG. 19B, an adjustment binding/locking lever 1916 may be part of a mechanism to lock the separation distance between the jaws once set. When used with needle electrodes, no additional feedback to the generator would be necessary since the e-field strength is set by the spacing between needle rows. If used with plate style electrodes, and in conjunction with sensor feedback to the generator, a precise e-field could be applied to any jaw gap.

Generally, as mentioned above, any of the elongate applicator tools (and/or clamps) described herein may be configured to determine the separation of the electrodes. In some examples having electrodes are on movable jaws one or more sensors and/or detector may be included to determine the separation between the jaws and therefore between the electrodes. Example sensors may include optical sensors, electrical sensors, mechanical sensors, or the like. The apparatus (e.g., tool) may include one or more sensors and/or detectors that directly measure the separation between the jaws and/or the electrodes (or sets of electrodes) or they may be configured to indirectly determine the spacing between the jaws and/or electrodes, for example, based on the setting of the control(s) controlling the spacing of the jaws. A detector may detect the control settings and or the relative positions of the jaws (e.g., the angle of the jaws in hinged/scissoring examples). Thus, in some examples, the spacing may be output as a function of the control settings for the jaws. A detector (optical detector, magnetic detector, mechanical detector, e.g., such as an encoder) may be used to detect or to determine the distance between the jaws and/or the electrodes either directly or indirectly. Alternatively or additionally, a sensor may be used, as described above. The determined distance may be provided from the detector and/or sensor of the elongate applicator apparatus (e.g., tool, clamp, laparoscope, etc.) to a controller, including a controller of or in communication with the pulse generator. Thus, any of these apparatuses (e.g., systems, devices, tools, including laparoscopes) described herein may be configured to measure the jaw and/or electrode distance on the apparatus (e.g., the elongate applicator tool) and use that measurement to set the applied energy (e.g., the voltage generated by the pulse generator).

The jaw distance may be measured by measuring the thickness of the tissue (e.g., tumor or other target tissue) and/or by measuring the gap or opening between the jaws. In some examples, the distal end region may include one or more markings that enable a user (e.g., clinician) to determine the jaw spacing and/or gap. The user may input the thickness into device (e.g., the elongate applicator tool) and/or the elongate applicator tool may communicate that thickness to the controller that sets the treatment voltage according to the thickness. Alternatively, in some examples the thickness may be input into a graphical user interface (GUI) for the pulse generator that may use the entered thickness to set the applied electric field, which may then be used to treat the patient.

Alternatively, in some examples the thickness of the target tissue is not directly measured, but instead the apparatus (e.g., the jaws) may be used to clamp down on the tissue (e.g., with the jaws), and when the jaws/electrodes are compressed on the tissue, the device may automatically measure jaw distance. For example, the measurement of jaw separation (and therefore electrode separation) or a predetermined jaw separation may be automatically sensed by the system/controller to adjust applied electric field. For example, one or more sensors (or detectors) may determine the thickness of the target tissue or jaw distance from a position of the jaws when placed on the patient. A controller, which in some cases may be included within the pulse generator, may use the determined thickness/jaw distance to cause the pulse generator to generate an appropriate electric field, e.g., at a target field strength. For example, the electric field may be configured to be between about 5-15 kV/cm to about 30 kV/cm. Given the distance between electrodes, to achieve a specific field, the apparatus can set a pulse voltage to achieve a target electric field strength of the pulsed electric field. Although the dimensions of the electrodes may be set, the operator (e.g., doctor) may enter a voltage. If the electrode dimensions (including in particular the separation between the electrodes) changes, the apparatus, such as the system 100, can measure the separation and automatically and appropriately adjust the pulse generator to generate an appropriate electric field.

Any of the apparatuses, including the elongate applicator tools and systems using them, may include one or more safety interlocking features to prevent the delivery of the high voltage, sub-microsecond pulsing until and unless the elongate applicator tool is properly deployed and in contact with a tissue, e.g., target tissue. For example, the methods and apparatuses described herein may be configured to emit one or a pattern of test pulses at very low power (e.g., low voltage) including at sub-microsecond rates to detect one or more properties of the electrical pathway including appropriate contact with a target tissue. In some examples, the apparatus may be configured to determine and detect the impedance at the one or more pairs of electrodes of the elongate applicator tool to confirm that the contact with the tissue (and the electrical pathway from the pulse generator to the tissue) are correct. Thus, these apparatuses and methods of use may include measuring an impedance of the tissue with the electrodes (e.g., surface electrodes, needle electrodes, knife electrodes, etc.). In some examples, the electrodes can be used to measure the impedance of the target tissue to be treated as well as the surrounding tissue. For example, electrical energy can be applied to the target tissue at a known frequency. In a first example, the electrical energy can initially be a low-voltage pulsed energy until the electrodes are positioned appropriately against or within the target tissue. This proper positioning can be confirmed with the impedance measurement. Once the electrodes are positioned within or against the target tissue, the electrical energy can comprise high voltage, fast pulsed energy, such as sub-microsecond pulses. However, it should be understood that any type of pulsed electrical energy can be applied to the target tissue (microsecond, nanosecond, picosecond, etc.).

During treatment of the tissue, treatment may continue if certain conditions are met, but may otherwise be terminated. For example, when a change in the impedance of the target tissue exceeds an impedance threshold, treatment may stop. Thus, the detection of contact and/or treatment may be ongoing during a treatment as well as before a treatment. For example, applying electrical energy to the tissue can change the impedance of the target tissue by breaking down the tissue itself. This change can be measured, and when the change in impedance exceeds an impedance threshold that indicates the tissue breakdown, the electrodes can be moved within the tissue or the treatment stopped. In another example, because the target tissue (e.g., tumor) may have different impedance from the surrounding tissue, a change in the impedance may occur because of the location of the elongate applicator tool and electrodes relative to the target tissue. Therefore, this change can be measured, and when the change in impedance exceeds an impedance threshold that indicates that location of the electrodes is outside the target tissue, the electrodes can be moved or the treatment stopped. The movement of electrodes can occur either during each pulse or in between pulses, or during entire application of the electric energy.

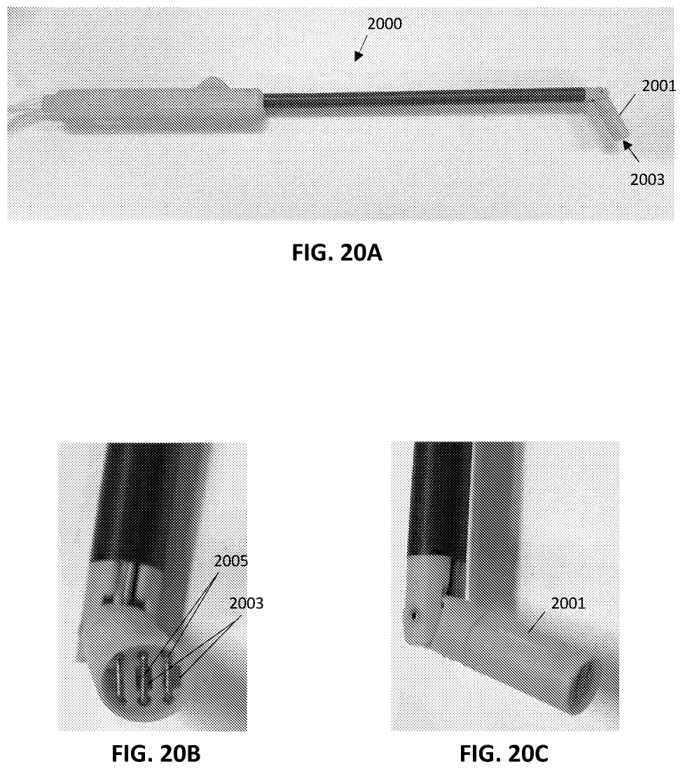

Any of the apparatuses described herein may be configured to include suction (e.g., vacuum) to assist in holding the electrodes to the tissue to be treated. Thus, in any of these apparatuses the electrodes may be adjacent to or surrounded by a suction or vacuum inlet. For example, FIG. 20A shows one example of an elongate applicator tool similar to that described above including an articulating tip 2001 and a vacuum inlet 2003 to assist with holding the electrodes on the tip in communication with the tissue to be treated. FIG. 20B shows an enlarged view of the articulating distal end, showing the vacuum tip with three electrodes 2005. In FIG. 20B, the electrodes are formed of wires or bars. Three vacuum inlets 2003 are shown behind and adjacent to each electrode. In this example, the vacuum is applied through these multiple vacuum inlets that are under and around the electrodes (e.g., needles and/or wires). Thus, the vacuum (suction) may pull the tissue onto the electrodes and/or may maintain tissue contact with the electrodes. This may remove air gaps between the electrodes and the tissue, which may reduce arcing and otherwise improve contact with the tissue. In any of these examples, the tip may include a sealing surface to channel the applied suction so that the seal may be maintained during treatment. In some examples the sealing surface may be formed of a soft silicon material on the tip. This soft silicone material may be formed as a cup or concavity to fit (e.g., seal) against the tissue.

In any of these examples the apparatus may include feedback to control the applied suction. For example, suction may be applied prior to the application of the energy (e.g., the high voltage, fast pulsed electrical energy). In some examples the suction may be automatically or manually applied before activating the application of the high voltage, fast pulsed electrical energy. Suction may be applied to a predetermined level to prevent damage to the tissue. Once the energy has been applied, the suction may be released, automatically or manually. In some examples, positive pressure may be applied to break any seal (e.g., to release the suction) and/or to separate the tissue from the electrodes. Positive pressure may be applied by applying fluid (e.g., saline) rather than air.

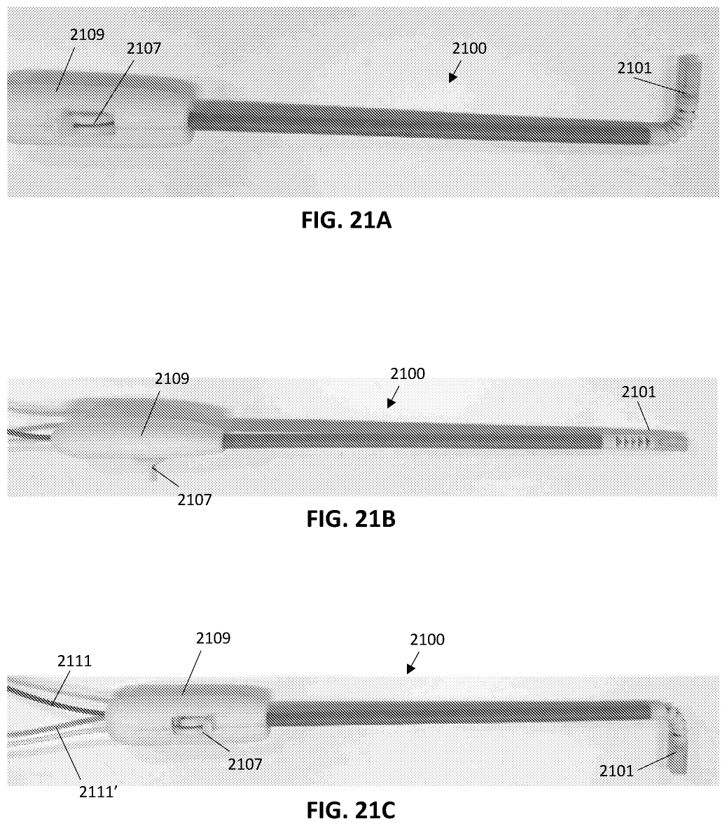

As shown in FIG. 20C, the example of the elongate applicator tool shown in FIGS. 20A-20B may be articulated from about 0 to about 90 degrees. The elongate applicator tool of FIGS. 20A-20C may be configured with a rigid shaft to be used as a laparoscopic instrument. In this embodiment, the elongate applicator tool may be inserted through a canula. In addition, the elongate applicator tool of FIGS. 20A-20C may be configured with a flexible shaft to be used as an endoscopic instrument or catheter. In such embodiments, the elongate applicator tool may be inserted through the working channel of an endoscope or catheter sheath. FIGS. 21A-21C illustrate another example of an elongate applicator tool in which the distal end region is articulating. In FIG. 21A the tip 2101 of the elongate applicator tool 2100 is articulated in a first direction by 90 degrees relative to the long axis of the elongate applicator tool. In FIG. 21A, the handle region 2109 includes a control 2107 for bending the tip 2101 of the apparatus as shown. FIG. 21B shows the same elongate applicator tool apparatus 2100 with the tip in a neutral, unbent, configuration. In FIG. 21C the apparatus 2100 is shown with the tip 2101 bent in an opposite direction from that shown in FIG. 21A. Any of these apparatuses may include one or more pressure (e.g., vacuum/suction) inlets at the distal end, as described above. In FIGS. 21B and 21C a pair of vacuum tubes (pressure lines) 2111, 2111′ extend from the proximal end of the apparatus for connection to a pressure (e.g., vacuum/suction) source and/or regulator. The elongate applicator tool of FIGS. 21A-21C may be configured with a rigid shaft to be used as a laparoscopic instrument. In this embodiment, the elongate applicator tool may be inserted through a canula. In addition, the elongate applicator tool of FIGS. 21A-21C may be configured with a flexible shaft to be used as an endoscopic instrument or catheter. In such embodiments, the elongate applicator tool may be inserted through the working channel of an endoscope or catheter sheath.

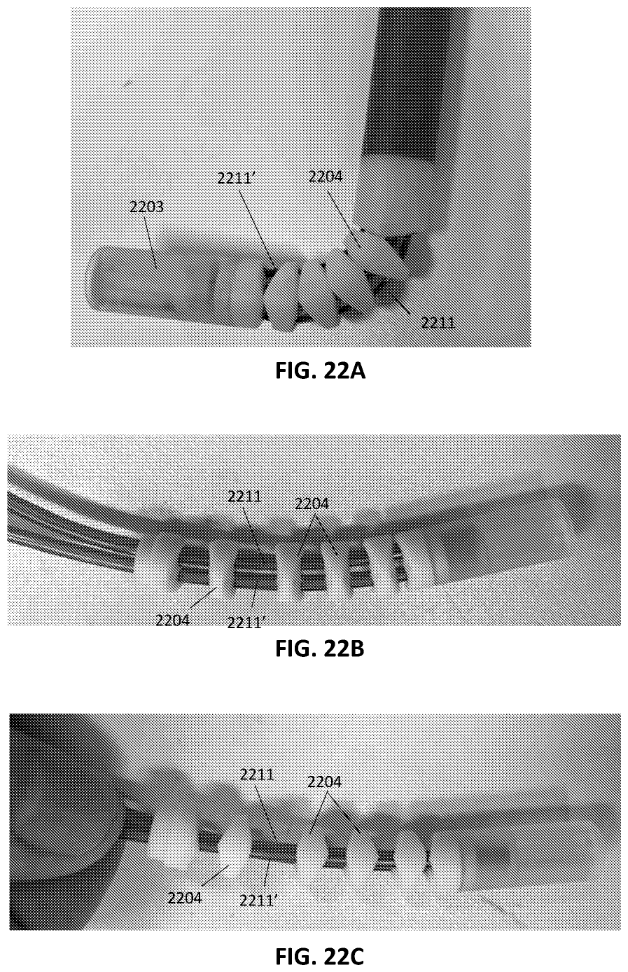

In some examples the pressure lines may be flexible and may be configured to help form the tip of the apparatus. For example, FIG. 22A shows one example of a tip (distal end region) of a elongate applicator tool including a distal end 2203 and a flexible or bending region, as described herein, with a pair of vacuum/suction lines (e.g., pressure lines 2211, 2211′); these lines may be used to apply pressure (negative or positive pressure), and also serve as the backbone of the bendable tip. In FIG. 22A, the tip may articulate by bending these ports. For example, as shown in FIG. 22A, the tip is coupled to the elongate body of the elongate applicator tool by a bending region formed by shaped vertebra 2204 (e.g., links, etc.) that are coupled together by the pressure line(s) 2211, 2211′. In some examples the pressure lines connect the vertebra, which are configured to steer the bending in a bending plane, as shown. A pull wire (or pair of pull wires) may be used to control the bending. Alternatively or additionally, the pressure lines may be used as pull wires to bend the tip. FIGS. 22B and 22C illustrate the assembly of a distal end region similar to that shown in FIG. 22A, showing the shaped vertebra 2204 riding over the pair of pressure lines 2211, 2211′.

In this example, the elongate applicator tool includes an articulating tip that uses a vacuum to assist with holding the electrodes on the treatment area. As described above, the articulating joint uses the flexibility of the vacuum tubing as the flexible hinges between segments. This may allow the tip to articulate over 180 degrees in a single plane, as shown in FIGS. 21A-21C. The apparatus can be configured with slightly different hinge segments to allow additional axis of articulation. The elongate applicator tool of FIGS. 22A-22C may be configured with a rigid shaft to be used as a laparoscopic instrument. In this embodiment, the elongate applicator tool may be inserted through a canula. In addition, the elongate applicator tool of FIG. 22A-22C may be configured with a flexible shaft to be used as an endoscopic instrument or catheter. In such embodiments, the elongate applicator tool may be inserted through the working channel of an endoscope or catheter sheath.

In general, a vacuum may be used with any electrodes (e.g., shaped bar, wire, or needle electrodes). As described, the vacuum may be applied through multiple holes under and/or around the electrodes to maintain consistent contact with tissue and to remove any air gaps and seal around the tissue. In some examples, the tips may incorporate soft silicon material (e.g., shaped like a suction cup) for better tissue holding function.

In general, in any of the apparatuses described herein the region of the tip near the electrodes may be formed of a non-conductive material, which may further enhance the safety and efficacy of these apparatuses, particularly when configured to apply high voltage, fast pulsed electrical energy. For example, the pull wires/tendons used to actuate the movement of the tip may be a non-conductive material, such as a polymeric material.

As mentioned above, any of the apparatuses described herein may be implemented in robotic systems that may be used to position and/or control the electrodes during a treatment. For example, a robotic system may include a movable (robotic) arm to which elongate applicator tool is coupled. Various motors and other movement devices may be incorporated to enable fine movements of an operating tip of the elongate applicator tool in multiple directions. The robotic system and/or elongate applicator tool may further include at least one image acquisition device (and preferably two for stereo vision, or more) which may be mounted in a fixed position or coupled (directly or indirectly) to a robotic arm or other controllable motion device. In some examples, the image acquisition device(s) may be incorporated into the elongate applicator tool.

### Example Methods

Also described herein are methods of treating tissue using any of the apparatuses described herein. For example, in general, an apparatus may be inserted into the patient's body through a natural channel (e.g., through the mouth, nose, etc.) or through an incision and the distal end region including the one or more jaws with one or more (e.g., a set of) electrodes on each jaw may be held against the tissue. The elongate body of the apparatus may be manipulated through the body to the target tissue to be treated. In some cases a single jaw may be used and held against the tissue so that a sub-microsecond (e.g., nanosecond) pulsed electric field may be applied through the electrodes on the jaw to treat the tissue. In some examples the tissue to be treated may be held within two (or more) jaws and the pulsed electric field may be applied between the electrodes on the jaws. The jaws may be articulated (opened/closed, and/or bent relative to the elongate body of the apparatus). One or more applications of energy may be made to treat the tissue. The treatment may include applying a predetermined amount of energy (e.g., a target or intended field strength).

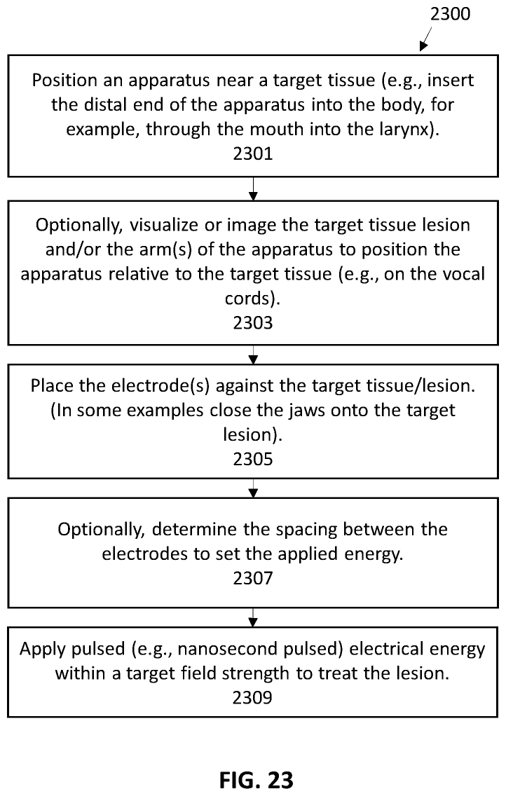

For example, any of these apparatuses may be used to treat a lesion (e.g., growths, such as nodules, polyps and/or cysts) on a vocal fold (e.g., vocal cord). FIG. 23 illustrates one example of a method 2300 of treating a lesion on a vocal cord. In FIG. 23, an apparatus for delivering a pulsed electric field (including a sub-microsecond or nanosecond pulse electric field) is positioned near the lesion (block 2301). Any appropriate apparatus may be used to remove or reduce a vocal cord lesion (e.g., polyp). The apparatus may include an elongate body (e.g., catheter body) that may be extended into the vocal cords, from an oral or nasal passage. The apparatus may include a distal end having one or more jaws (e.g., two jaws) with one or more electrodes on each jaw. The one or more jaws may move relative to the other jaw(s) to open and close. In some examples only one jaw moves. In examples in which the apparatus includes a pair of jaws, the jaws may be positioned on either side of the lesion. In some cases, the jaws may be articulated both open/closed and may be bent or angled relative to the elongate body of the apparatus. Optionally, the lesion and/or the jaws may be visualized (block 2303), for example, using a scope (such as a fiber optic scope) that is passed through or alongside the apparatus. In some examples the apparatus includes one or more channels for imaging.

The apparatus may have side facing, non-penetrating electrodes (e.g., plate electrodes, disc electrodes, etc.) that may contact the lesion tissue. The jaws may be closed onto the lesion so that the electrodes (or sets of electrodes) are placed on either side of the lesion (block 2305). In some cases the lesion may be compressed between the jaws. In some implementations of the method, the spacing between the electrodes may be determined (block 2307) (e.g., using a detector or sensor in the apparatus) and this spacing may be used by a controller to set (by controlling the pulse generator) the applied energy so that a pulse electric field having a target field strength may be applied to the lesion. In any of these examples, nanosecond pulses (e.g., pulses having a duration of between about 1 ns and 1 ms, e.g., between 1 ns and 950 ns, between 1 ns and 900 ns, between 1 ns and 750 ns, between 1 ns and 600 ns, etc.) having a field strength of between 5 kV/cm to 100 kV/cm (e.g., between 10 kV/cm and 100 kV/cm, etc.) may be applied to treat the lesion (block 2309).

In some examples only a single jaw may be used. The jaw may include multiple electrodes (e.g., side-facing, non-penetrative electrodes). The apparatus may be maneuvered to the tissue to be treated, such as a lesion within the vocal cords, and the jaw (e.g., the electrodes on the jaw) may be driven or held against the target tissue so that sub-microsecond pulsed energy may be applied as described above.

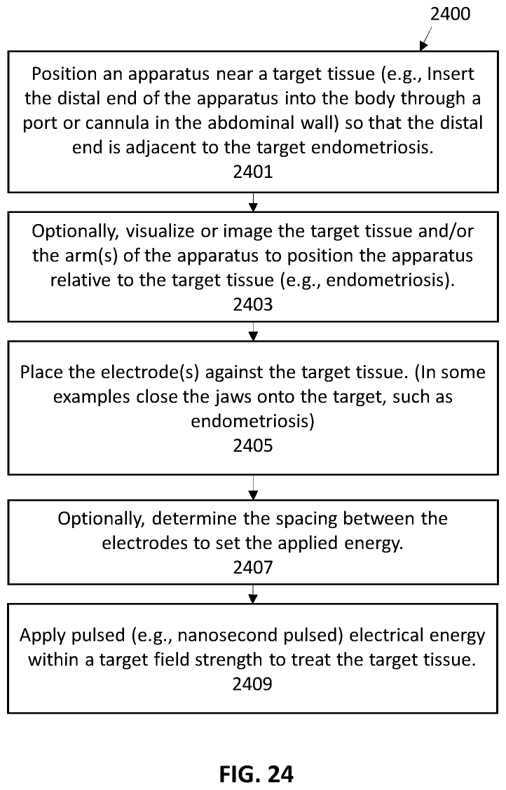

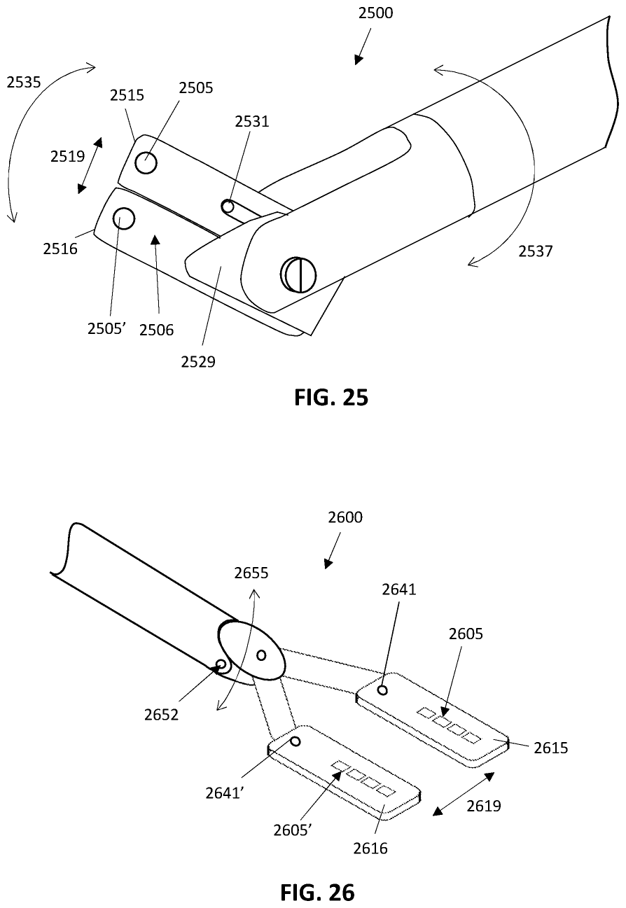

Also described herein is an example of a method 2400 of treating endometriosis using an apparatus as described herein, as illustrated in FIG. 24. For example, an apparatus for delivering a pulsed electric field (including a sub-microsecond or nanosecond pulse electric field) may be used to treat endometriosis, e.g., to remove or reduce. The apparatus may be positioned near the target tissue, for example, inserted into the body (e.g., through a port of cannula in an abdominal wall and/or through a natural orifice) so that the distal end of the apparatus is adjacent to the target tissue (block 2401). As described above, an apparatus may include an elongate body (e.g., catheter body) that may be extended into the body of the subject, such as through a port or cannula in the abdominal wall. In some examples the apparatus may be inserted at least partially through a natural orifice (e.g., within the female urogenital system such as the vaginal canal) so that the distal end of the apparatus, including the electrodes, may be positioned adjacent to the tissue to be treated (e.g., at the fallopian tubes, ovaries, intestines, pelvic lining, etc.). The apparatus may include a distal end having one or more jaws (e.g., two jaws) with one or more electrodes on each jaw. The one or more jaws may move relative to the other jaw(s) to open and close. In some examples only one jaw moves. In examples in which the apparatus includes a pair of jaws, the jaws may be positioned on either side of the tissue to be treated. However, when a target tissue is a flat surface, both jaws may be positioned on the same side of the target tissue, as described below in reference to FIGS. 25-26. In some cases, the jaws may be articulated both open/closed and may be bent or angled relative to the elongate body of the apparatus. Optionally, the target tissue, such as abnormal endometrial lining, and/or the jaws may be visualized (block 2403), for example, using a scope (such as a fiber optic scope) that is passed through or alongside the apparatus to image the target tissue and/or the jaw(s). In some examples the apparatus includes one or more channels for imaging.

The apparatus may have side facing, non-penetrating electrodes (e.g., plate electrodes, disc electrodes, etc.) that may contact the lesion or target tissue (block 2405). The jaws may be closed onto the target tissue so that the electrodes (or sets of electrodes) are placed on either side of the target tissue to be treated. In some cases the target tissue may be compressed between the jaws. In some implementations, the spacing between the electrodes may be determined (e.g., using a detector or sensor in the apparatus) and this spacing may be used by a controller to set (by controlling the pulse generator) the applied energy so that a pulse electric field having a target field strength may be applied to the lesion (block 2407). Alternatively or additionally the apparatus may adjust the spacing between the electrodes so that the applied energy may be within a target range. In any of these examples, nanosecond pulses (e.g., pulses having a duration of between about 1 ns and 1 ms, e.g., between 1 ns and 950 ns, between 1 ns and 900 ns, between 1 ns and 750 ns, between 1 ns and 600 ns, etc.) having a field strength of between 5 kV/cm to 100 kV/cm (e.g., between 10 kV/cm and 100 kV/cm, etc.) may be applied to treat the target tissue (block 2409).

In examples in which a single jaw is used, the jaw may include multiple electrodes (e.g., side-facing, non-penetrative electrodes). The apparatus may be maneuvered to the tissue to be treated, such as adjacent to the target endometriosis (e.g., the target abnormal endometrial tissue), and the jaw (e.g., the electrodes on the jaw) may be driven or held against the target tissue so that sub-microsecond pulsed energy may be applied as described above.

The methods illustrated in FIGS. 23 and 24 may be performed using any of the apparatuses described herein, including, for example, apparatuses including side-facing, non-penetrative electrodes. For example, the apparatuses described herein may be configured so that the electrodes are all on the same side of each of the jaws. This configuration may allow the electrodes (or sets of electrodes) to be positioned against the target tissue (especially, when the target tissue has a substantially flat surface that cannot be easily grasped), such as, e.g., target tissue affected by endometriosis, and the spacing between the electrodes may be adjusted without clamping the target tissue between them. For example, FIG. 25 illustrates another example of an elongate applicator apparatus (e.g., applicator tool) similar to that shown in FIGS. 5A-5D, above. However, in FIG. 25, all of the electrodes 2505, 2505′ are on the same face (which may be referred to herein as a primary face 2506) of the jaws. In FIG. 25 the primary face of the jaws faces out (e.g., in this illustration, out of the page), so that both jaws and the electrodes on the jaws may be held against the tissue to be treated. The elongate applicator tool of FIG. 25 may be configured as a laparoscope or an endoscope (or for use with a laparoscope, endoscope and/or a catheter). The elongate applicator tool of FIG. 25 may be configured with a rigid shaft to be used as a laparoscopic instrument. In this embodiment, the elongate applicator tool may be inserted through a canula. In addition, the elongate applicator tool of FIG. 25 may be configured with a flexible shaft to be used as an endoscopic instrument or catheter. In such embodiments, the elongate applicator tool may be inserted through the working channel of an endoscope or catheter sheath. In FIG. 25 the elongate applicator tool 2500 may include a pair of jaws (upper 2515, lower 2516) that may open and close to adjust the spacing between the first electrode 2505 (or in some examples, set of electrodes) and a second electrode 2505′ (or set of electrodes). Opening and closing of the jaws may be controlled from the proximal end of the elongate applicator tool (not shown). In some examples, the opening and closing of the jaws may be controlled through a control (e.g., knob, etc.) located on or near a handle coupled to, or part of, the elongate applicator tool. In some other examples, the opening and closing of the jaws may be controlled directly or indirectly by a processor, state machine, embedded controller, or any other component, such as controller, including one or more processors. As in any of the apparatuses described herein, the spacing between the jaws may be determined and/or controlled. The spacing may be determined directly, e.g., using one or more sensors or detectors, or indirectly, e.g., inferred based on calibration of the control or controls moving the jaws. Although FIG. 25 shows the jaws closed, with the distance between the electrodes (or sets of electrodes) 2519 at a minimum, the jaws may be opened similar to the example shown in FIGS. 5A-5D. Similarly, the distal end region of the elongate applicator apparatus shown in FIG. 25 may also be articulated by rotating up or down 2535. The rotation of the jaws may be independent of the opening and closing of the jaws. In some examples, the distal end region may be pivoted along the long axis 2537. These various degrees of freedom allow the electrodes to be properly presented against the target tissue.

As mentioned, one or more electrodes 2505, 2505′ may be coupled with each jaw, and/or in some examples the jaws may contain or themselves form the electrodes (e.g., plate or disc electrodes). As mentioned, the electrodes may be non-penetrative (as shown in FIG. 25) or penetrative, such as needle electrodes.

The apparatus may include a jaw actuating mechanism that is configured to allow movement of the upper 2515 and lower 2516 jaws relative to each other. Either both jaws may be moved to open/close, or one jaw may move relative to the other, stationary, jaw. In FIG. 25, (as described above for FIGS. 5A-5B) the jaws may be configured to open and close substantially in parallel, e.g., by rotating a pair of eccentric tabs 2529 with side pins 2531 that are configured to be rotated in opposite directions (e.g., clockwise, CW, and counterclockwise, CCW) to cause the jaws move relative to each other.

FIG. 26 illustrates another example of an elongate applicator apparatus 2600 that includes two or more side-facing electrodes 2605, 2605′ facing the same (e.g. primary side) of the elongate applicator apparatus. In FIG. 26, scissoring jaws or arms 2615, 2616 are configured as paddle-like jaws that may open and close to increase or decrease the spacing between the electrodes, as shown by arrow 2619. The jaws in this example each include a hinge joint 2641, 2641′ so that the scissoring movement, which may be proximally actuated, results in a parallel opening and closing of the jaws. The distal end region including the jaws may also be moved in two or more degrees of freedom about a wrist region 2652. For example in FIG. 26 the apparatus includes a two degree-of-freedom wrist so that the electrodes can be presented against the tissue. In some examples the distal end may also rotate about the long axis of the apparatus. Thus, the apparatus shown in FIG. 26 is another example including side-facing, non-penetrative electrodes having spacing that may be adjusted.

Thus, described herein are apparatuses for delivering a high voltage, sub-microsecond pulsed electric field that include side-facing electrodes having adjustable spacing. For example, an apparatus for delivering a high voltage, sub-microsecond pulsed electric field may include an elongate shaft, a distal end region comprising a first jaw and a second jaw, the distal end region extending from a distal end of the elongate shaft, a first electrode (or set of electrodes) on a first side of the first jaw and a second electrode (or second set of electrodes) on a second side of the second jaw, wherein the first side and the second side face the same direction. The apparatus may also include a proximal handle including a control for opening and closing the first jaw and the second jaw, and a spacing control configured to adjust an electrode spacing distance between the first electrode and the second electrode.

The first side and the second side may both be on the primary side (e.g., left-facing, right facing, facing up or facing down) relative to the distal end of the apparatus. Thus, the first electrode (or set of electrodes) and the second electrode (or set of electrodes) may be applied against the same side of the tissue. The side-facing electrodes may be non-penetrating electrodes.

The spacing between the first electrode (or set of electrodes) and the second electrode (or set of electrodes) may be adjusted by adjusting the separation of the jaws. In any of the apparatuses described herein, the jaws may be configured so that they remain parallel during opening and closing. Any of these apparatuses, including the side-facing electrode examples such as those shown in FIGS. 25 and 26, above, may include a proximal handle including a spacing control for adjusting a spacing between the first electrode and the second electrode. The spacing control may control the opening and closing of the jaws. Furthermore, any of these apparatuses may include an articulation control, for example, on the proximal handle configured to adjust the bend angle of the distal end region including the jaws, and/or a rotation control on the proximal region (e.g., proximal handle) configured to control axial rotation of the distal end region (including the jaws) of the apparatus.

As described in greater detail above, any of these apparatuses may include imaging, including one or more ports or channels extending through the apparatus for passing an imaging device, such as a fiber optic device, camera, etc. In general, any of the apparatuses described herein may be configured as a laparoscopic apparatus or as an endoscopic apparatus. For example, any of these apparatuses may include one or more cameras in addition to the one or more jaws and the electrodes. Thus, any of the elongate applicator apparatuses (e.g., elongate applicator tools) described herein may be configured with a rigid shaft to be used as a laparoscopic instrument and may be configured to be (and may be) inserted through a canula. Alternatively or additionally, any of the elongate applicator apparatuses (e.g., elongate applicator tools) described herein may be configured with a flexible shaft to be used as an endoscopic instrument or catheter and may be inserted through the working channel of an endoscope or catheter sheath.

Embodiments of the methods of the present disclosure may be implemented using computer software, firmware or hardware. Various programming languages and operating systems may be used to implement the present disclosure. The program that runs the method and system may include a separate program code including a set of instructions for performing a desired operation or may include a plurality of modules that perform such sub-operations of an operation or may be part of a single module of a larger program providing the operation. The modular construction facilitates adding, deleting, updating and/or amending the modules therein and/or features within the modules.

In some embodiments, a user may select a particular method or embodiment of this application, and the processor will run a program or algorithm associated with the selected method. In certain embodiments, various types of position sensors may be used. For example, in certain embodiment, a non-optical encoder may be used where a voltage level or polarity may be adjusted as a function of encoder signal feedback to achieve a desired angle, speed, or force.

Certain embodiments may relate to a machine-readable medium (e.g., computer readable media) or computer program products that include program instructions and/or data (including data structures) for performing various computer-implemented operations. A machine-readable medium may be used to store software and data which causes the system to perform methods of the present disclosure. The above-mentioned machine-readable medium may include any suitable medium capable of storing and transmitting information in a form accessible by processing device, for example, a computer. Some examples of the machine-readable medium include, but not limited to, magnetic disc storage such as hard disks, floppy disks, magnetic tapes. It may also include a flash memory device, optical storage, random access memory, etc. The data and program instructions may also be embodied on a carrier wave or other transport medium. Examples of program instructions include both machine code, such as produced by a compiler, and files containing higher level code that may be executed using an interpreter.

Any of the methods (including user interfaces) described herein may be implemented as software, hardware or firmware, and may be described as a non-transitory computer-readable storage medium storing a set of instructions capable of being executed by a processor (e.g., computer, tablet, smartphone, etc.), that when executed by the processor causes the processor to perform or control performing of any of the steps, including but not limited to: displaying, communicating with the user, analyzing, modifying parameters (including timing, frequency, intensity, etc.), determining, alerting, or the like. In some exemplary embodiments hardware may be used in combination with software instructions to implement the present disclosure.

When a feature or element is herein referred to as being “on” another feature or element, it can be directly on the other feature or element or intervening features and/or elements may also be present. In contrast, when a feature or element is referred to as being “directly on” another feature or element, there are no intervening features or elements present. It will also be understood that, when a feature or element is referred to as being “mounted”, “connected”, “attached” or “coupled” to another feature or element, it can be directly mounted, connected, attached or coupled to the other feature or element or intervening features or elements may be present. In contrast, when a feature or element is referred to as being “directly mounted”, “directly connected”, “directly attached” or “directly coupled” to another feature or element, there are no intervening features or elements present. Although described or shown with respect to one embodiment, the features and elements so described or shown can apply to other embodiments. It will also be appreciated by those of skill in the art that references to a structure or feature that is disposed “adjacent” another feature may have portions that overlap or underlie the adjacent feature.

Terminology used herein is for the purpose of describing particular embodiments only and is not intended to be limiting. For example, as used herein, the singular forms “a”, “an” and “the” are intended to include the plural forms as well, unless the context clearly indicates otherwise. As used herein, the term “and/or” includes any and all combinations of one or more of the associated listed items.

Spatially relative terms, such as “under”, “below”, “lower”, “over”, “upper” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if a device in the figures is inverted, elements described as “under” or “beneath” other elements or features would then be oriented “over” the other elements or features. Thus, the exemplary term “under” can encompass both an orientation of over and under. The device may be otherwise oriented (rotated 90 degrees or at other orientations) and the spatially relative descriptors used herein interpreted accordingly. Similarly, the terms “upwardly”, “downwardly”, “vertical”, “horizontal” and the like are used herein for the purpose of explanation only unless specifically indicated otherwise.

Although the terms “first” and “second” may be used herein to describe various features/elements (including steps), these features/elements should not be limited by these terms, unless the context indicates otherwise. These terms may be used to distinguish one feature/element from another feature/element. Thus, a first feature/element discussed below could be termed a second feature/element, and similarly, a second feature/element discussed below could be termed a first feature/element without departing from the teachings of the present apparatuses and methods.

The terms “comprises” and/or “comprising,” when used in this specification (including the claims), specify the presence of stated features, steps, operations, elements, and/or components, but do not preclude the presence or addition of one or more other features, steps, operations, elements, components, and/or groups thereof. Unless the context requires otherwise, “comprise”, and variations such as “comprises” and “comprising,” means various components can be co-jointly employed in the methods and articles (e.g., compositions and apparatuses including device and methods). For example, the term “comprising” will be understood to imply the inclusion of any stated elements or steps but not the exclusion of any other elements or steps.

Any of the apparatuses and methods described herein may include all or a sub-set of the components and/or steps, and these components or steps may be either non-exclusive (e.g., may include additional components and/or steps) or in some variations may be exclusive, and therefore may be expressed as “consisting of” or alternatively “consisting essentially of” the various components, steps, sub-components or sub-steps.

As used herein in the specification and claims, including as used in the examples and unless otherwise expressly specified, all numbers may be read as if prefaced by the word “about” or “approximately,” even if the term does not expressly appear. The phrase “about” or “approximately” may be used when describing magnitude and/or position to indicate that the value and/or position described is within a reasonable expected range of values and/or positions. For example, a numeric value may have a value that is +/−0.1% of the stated value (or range of values), +/−1% of the stated value (or range of values), +/−2% of the stated value (or range of values), +/−5% of the stated value (or range of values), +/−10% of the stated value (or range of values), etc. Any numerical values given herein should also be understood to include about or approximately that value unless the context indicates otherwise. For example, if the value “10” is disclosed, then “about 10” is also disclosed. Any numerical range recited herein is intended to include all sub-ranges subsumed therein. Although various illustrative embodiments are described above, any of a number of changes may be made to various embodiments without departing from the scope of the disclosure as described by the claims. For example, the order in which various described method steps are performed may often be changed in alternative embodiments, and in other alternative embodiments one or more method steps may be skipped altogether. Optional features of various device and system embodiments may be included in some embodiments and not in others. Therefore, the foregoing description is provided primarily for exemplary purposes and should not be interpreted to limit the scope of the apparatuses and methods as it is set forth in the claims.

Various embodiments may be referred to herein individually or collectively by the term “invention” merely for convenience and without intending to voluntarily limit the scope of this application to any single invention or inventive concept, if more than one is, in fact, disclosed. Thus, although specific embodiments have been illustrated and described herein, any arrangement calculated to achieve the same purpose may be substituted for the specific embodiments shown. This disclosure is intended to cover any and all adaptations or variations of various embodiments. Combinations of the above embodiments, and other embodiments not specifically described herein, will be apparent to those of skill in the art upon reviewing the above description.

1.-40. (canceled)

41. A system for delivering a sub-microsecond pulsed electric field, the system comprising:
an elongate applicator tool comprising:
an elongate shaft;
a distal end region comprising a first jaw and a second jaw, the distal end region extending from a distal end of the elongate shaft;
a first side-facing electrode on the first jaw and a second side-facing electrode on the second jaw, wherein the first side-facing electrode and the second side-facing electrode face a same direction;
a high voltage connector; and
a pulse generator configured to generate a plurality of electrical pulses having an amplitude of at least 0.1 kV and a duration of less than 1000 nanoseconds, the pulse generator comprising a port configured to connect to the high voltage connector.

42. The system of claim 41, further comprising a proximal handle including one or more controls configured to control opening and closing of the first jaw and the second jaw and/or articulation of the distal end region.

43. The system of claim 41, further comprising a spacing control configured to adjust a spacing between the first side-facing electrode and the second side-facing electrode.

44. The system of claim 43, wherein the spacing control is configured to adjust the spacing between the first side-facing electrode and the second side-facing electrode from between about 0.5 mm to 1 mm or more.

45. The system of claim 41, further comprising a jaw spacing control configured to set a jaw spacing distance between the first side-facing electrode and the second side-facing electrode in a closed configuration.

46. The system of claim 45, further comprising a sensor configured to detect the jaw spacing distance between the first side-facing electrode and the second side-facing electrode.

47. The system of claim 42, wherein the one or more controls comprising a control configured to extend and retract the first and second side-facing electrodes from a housing.

48. The system of claim 41, wherein the elongate applicator tool is configured as a laparoscopic instrument, an endoscopic instrument, or a catheter, or for use through a channel of a laparoscope, an endoscope, or a catheter.

49. The system of claim 41, wherein the first jaw and the second jaw are configured to open in parallel.

50. The system of claim 41, further comprising a proximal handle configured to be coupled to a robotic arm for computer controlled activation and/or positioning of the first and second side-facing electrodes.

51. The system of claim 41, further comprising a controller configured to adjust the plurality of electrical pulses from the pulse generator based, at least in part, on one of: a detected spacing between the first side-facing electrode and the second side-facing electrode; and a user provided spacing between the first side-facing electrode and the second side-facing electrode.

52. The system of claim 41, wherein the first and second side-facing electrodes are non-penetrating electrodes, penetrating electrodes, or a combination thereof.

53. An apparatus for delivering a high voltage, sub-microsecond pulsed electric field, the apparatus comprising:
an elongate shaft;
a distal end region comprising a first jaw and a second jaw, the distal end region extending from a distal end of the elongate shaft;
a first side-facing electrode on the first jaw;
a second side-facing electrode on the second jaw, wherein the first side-facing electrode and the second side-facing electrode face a same direction so that they may be positioned against a same side of a target tissue;
a proximal handle;
a control for opening and closing the first jaw and the second jaw; and
a spacing control configured to adjust an electrode spacing distance between the first side-facing electrode and the second side-facing electrode.

54. The apparatus of claim 53, further comprising a sensor configured to determine the electrode spacing distance between the first electrode and the second electrode.

55. The apparatus of claim 53, wherein the distal end region is configured to be controllably bent to a bend angle relative to the elongate shaft and wherein the proximal handle comprises a control for selecting the bend angle.

56. The apparatus of claim 53, wherein the first side-facing electrode and the second side-facing electrode comprise needle electrodes, knife electrodes or other penetrating electrodes.

57. The apparatus of claim 53 wherein the first side-facing electrode and the second side-facing electrode comprise surface electrodes, plate electrodes or other non-penetrating electrodes.

58. The apparatus of claim 53, further comprising a controller configured to receive the electrode spacing distance between the first side-facing electrode and the second side-facing electrode, and to adjust an applied pulsed electric field based, at least in part, on the electrode spacing distance between the first side-facing electrode and the second side-facing electrode.

59. The apparatus of claim 53, further comprising a suction inlet adjacent to the first and second side-facing electrodes.

60. The apparatus of claim 53, wherein the proximal handle is configured to be coupled to a robotic arm for computer controlled activation and/or positioning of the first and second electrodes.

61. A method comprising:
detecting or setting a separation distance between a first side-facing electrode and a second side-facing electrode at a distal end region of an elongate applicator tool, the first side-facing electrode and the second side facing electrode contact a same side of a target tissue when the distal end region of the elongate applicator tool is placed against the target tissue;
determining, based at least in part on the separation distance between the first and the second side-facing electrodes, an energy to be applied to the target tissue in a plurality of electrical pulses having an amplitude of greater than 0.1 kV and a duration of less than 1000 nanoseconds, to treat the target tissue; and
applying the energy to the first side-facing electrode and the second side-facing electrode.

62. The method of claim 61, further comprising adjusting or allowing to adjust the separation distance between the first side-facing electrode and the second side-facing electrode before or after detecting the separation distance between the first and second side-facing electrodes and/or transmitting the separation distance to a pulse generator.

63. The method of claim 61, wherein the first side-facing electrode is on a first jaw and a second side-facing electrode is on a second jaw, the method further comprising setting or allowing to set a closing distance between the first jaw and the second jaw.

64. The method of claim 61, the method further comprising applying suction to seal the first and second side-facing electrodes to the target tissue while delivering the energy.
